(19) United States
(12) Patent Application Publication
(10) Pub. No.: US 2026/0022113 A1
(43) Pub. Date: Jan. 22, 2026

(54) Process for the Preparation of Pyroxasulfone

(71) Applicant: Atticus, LLC, Cary, NC (US)

(72) Inventors: Vidyanatha Anantha Prasad, Kansas City, KS (US); Binlong Liu, Beijing (CN); Youzhi Chen, Beijing (CN); Shannon Russell, Raleigh, NC (US)

(21) Appl. No.: 19/224,775

(22) Filed: May 31, 2025

Related U.S. Application Data
(60) Provisional application No. 63/654,627, filed on May 31, 2024.

(51) Int. Cl. C07D 413/12

(52) U.S. Cl. CPC C07D 413/12

(57) ABSTRACT

The present invention relates to a process for the preparation of pyroxasulfone of Formula I or salt thereof. More particularly, the present invention relates to an improved process for the preparation of pyroxasulfone or salt thereof, wherein the oxidation step to prepare pyroxasulfone is performed under reaction conditions using one or more glymes as a solvent, particularly without a metal catalyst and in the presence of a carboxylic acid, and where the reaction may be performed using a continuous-flow procedure.

### CROSS-REFERENCE TO RELATED APPLICATIONS

This application claims the benefit of priority from U.S. Provisional Patent Application No. 63/654,627, filed May 31, 2024. The content of the aforementioned application, including any intervening amendments thereto, is hereby incorporated by reference in its entirety.

### FIELD OF THE INVENTION

The present invention relates to a process for the preparation of pyroxasulfone of Formula I or salt thereof. More particularly, the present invention relates to an improved process for the preparation of pyroxasulfone or salt thereof, wherein the oxidation step to prepare pyroxasulfone is performed under reaction conditions using one or more glymes as a solvent or part of a solvent system, in particular in the absence of a metal catalyst and in the presence of a carboxylic acid. The improved process can be performed as a batch process, a continuous-flow process, or a combination thereof.

### BACKGROUND OF THE INVENTION

Pyroxasulfone is an herbicide belonging to the isoxazoline class of herbicides. Pyroxasulfone inhibits fatty acid synthesis. The primary chemical name of pyroxasulfone is 3-[5-(difluoromethoxy)-1-methyl-3-(trifluoromethyl)-1H-pyrazol-4-ylmethylsulfonyl]-4,5-dihydro-5,5-dimethylisoxazole. The structure of pyroxasulfone can be represented as shown in Formula I:

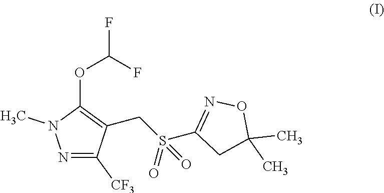

Pyroxasulfone and salts thereof are disclosed in U.S. Pat. No. 7,238,689, among other patents and publications. Pyroxasulfone is a pre-emergence herbicide for treating wheat, corn, and soybean, among other crops and plants. Pyroxasulfone inhibits the biosynthesis of very-long-chain fatty acids in plants and has shown excellent herbicidal activity against grass and broadleaf weeds at lower application rates compared to other commercial herbicides. Pyroxasulfone is also used as a pre-emergence herbicide to control grass and small-seeded broadleaf weeds. In fields of genetically modified crops, pyroxasulfone controlled weeds that were resistant to non-selective herbicides. Pyroxasulfone has been classified in the Herbicide Resistance Action Committee Group K3.

There are several known processes of making pyroxasulfone, although the known processes have various limitations and shortcomings. Processes for making pyroxasulfone are disclosed, for example, in WO2023017542A1, US20120264947, and WO2020240392A1.

For instance, current procedures for the conversion of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole (Formula II) to pyroxasulfone typically employ the following reagents/catalysts: acetic acid, sulfuric acid, sodium tungstate, and/or a combination thereof, during the oxidation step with aqueous hydrogen peroxide in a solvent such as toluene at 90 to 110° C. Typically, in current processes, 6 to 7 moles of aqueous hydrogen peroxide are used per mole of the compound of Formula II, with long reaction times of 12 to 15 hours. This current process is generally the process that is preferentially performed on an industrial scale. Furthermore, typical procedures for making pyroxasulfone employ a batch process for the various steps of making the final product.

There remains a need for improved processes of making pyroxasulfone. In particular, there remains a need for an improved process for making pyroxasulfone using solvents that result in higher yield and improved purity profiles of the end product and for processes that can incorporate continuous-flow methods.

### SUMMARY OF THE INVENTION

According to the present invention, an improved process is provided for the preparation of pyroxasulfone and salts thereof. The improved process, more specifically, provides for the preparation of pyroxasulfone (Formula I) by the oxidation of the sulfide compound of Formula II (3-(5-(Difluoromethoxy)-1-methyl-3-(trifluoromethyl)-1H-pyrazol-4-yl)methylsulfanyl-4H-5,5-dimethylisoxazole) in a solvent that contains one or more glymes in the absence of a metal catalyst, in the presence of a carboxylic acid, in which the reaction may further be performed using a continuous-flow process.

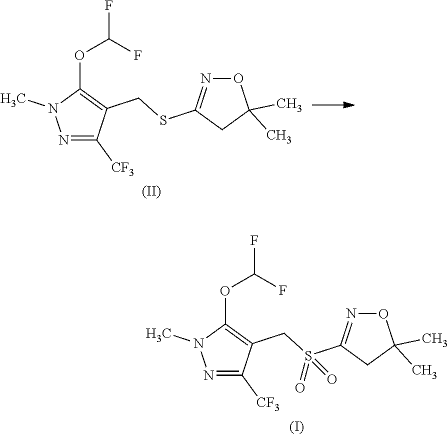

The use of one or more glymes as the solvent or in a solvent provides an improved method of making pyroxasulfone. The process generally includes the process of reacting the compound of Formula II with a suitable oxidizing agent in a solvent that comprises one or more glymes to obtain pyroxasulfone (Formula I). Other aspects of the invention are described in further detail herein.

### BRIEF DESCRIPTION OF THE DRAWINGS

FIG. 1 depicts the flow diagram of a process for preparing pyroxasulfone comprising (5,5-dimethyl(4,5-dihydroisoxazolo-3-yl))thiocarboxamidine hydrobromide and 4-chloromethyl-5-difluoromethoxy-1-methyl-3-trifluoromethyl-pyrazole as starting materials in a batch process, followed by the oxidation using a continuous-flow process to prepare pyroxasulfone.

FIG. 2 depicts a flow diagram of a process for preparing pyroxasulfone using (1-methyl-3-(trifluoromethyl)-1H-pyrazol-5-ol) as a starting material that progresses through several reactions before being reacting with 5,5-dimethyl-4,5-dihydroisoxazol-3-yl-carbamimidothioate to form a compound of Formula II, with all steps involved occurring under batch or continuous-flow conditions. The compound of Formula II is then oxidized using a continuous-flow process to prepare pyroxasulfone.

FIG. 3 depicts a flow diagram of a process of preparing pyroxasulfone starting from 1-methyl-3-(trifluoromethyl)-1H-pyrazol-5-ol as a starting material that progresses through several reactions in a batch process before being reacted with 3-chloro-5, 5-dimethyl-4, 5-dihydroisoxazole in a continuous-flow process to form a compound of Formula II. The compound of Formula II is then oxidized using a continuous-flow process to prepare pyroxasulfone.

FIG. 4 depicts a flow diagram of a process for preparing pyroxasulfone using 4-(((4,5-dihydro-5,5-dimethyl-3-isoxazolyl)thio)methyl)-1-methyl-3-(trifluoromethyl)-1H-pyrazol-5-ol and a fluorinated methyl halide in a continuous-flow process using a continuous-stirred tank reactor (“CSTR”). This is followed by oxidation in a continuous-flow process using a tubular reactor to prepare pyroxasulfone.

FIG. 5 depicts a flow diagram of a process for preparing pyroxasulfone using 3-chloro-5,5-dimethyl-4,5-dihydroisoxazole and thioacetic acid S-(5-difluoromethoxy-1-methyl-3-trifluoromethyl-1H-pyrazol-4-ylmethyl)-ester as starting materials in a batch process to form a compound of Formula II. The compound of Formula II is then oxidized in a continuous-flow process using a continuous-stirred tank reactor to form pyroxasulfone.

FIG. 6 depicts a flow diagram of a process for preparing pyroxasulfone using a compound of Formula II and hydrogen peroxide in a continuous-flow process in which a continuous-stirred tank reactor is heated using induction heating on steel beads. The product of the reaction is collected using a sample loop for offline analysis using for example, HPLC.

FIG. 7 depicts a flow diagram of a process for preparing pyroxasulfone using a compound of Formula II and hydrogen peroxide in a continuous-flow process in which the compound of Formula II is pumped at a rate of 4.0 g/min and the hydrogen peroxide is pumped at a rate of 0.8 g/min using feed pumps. The reactants enter a tubular reactor held at 90-95° C. for a three-hour residence time to form pyroxasulfone. An outlet (such as a sample loop) is used to collect the reaction stream for offline and/or realtime analysis using HPLC.

FIG. 8 depicts a flow diagram of a process for preparing pyroxasulfone using 1-methyl-3-(trifluoromethyl)-1H-pyrazol-5-ol as a starting material. (1-methyl-3-(trifluoromethyl)-1H-pyrazol-5-ol) proceeds through successive continuous-flow reactions with formaldehyde and sodium hydroxide, chlorodifluoromethane, chlorinating agent, 5,5-dimethyl-4,5-dihydroisoxazol-3-yl-carbamimidothioate, and hydrogen peroxide in a series of tubular reactors in which the reagents are pumped into the reactors using feed pumps.

### DETAILED DESCRIPTION OF THE INVENTION

### Definitions

For convenience, before providing a further description of the present invention, certain terms employed in the specification and examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

The term “about” or “approximately” as used herein is inclusive of the stated value and means within an acceptable range of deviation for a particular value, as determined by one of ordinary skill in the art, considering the measurement in question and the error associated with measurement of the particular quantity, that is, the limitations of the measurement system. For instance, “about” can mean within one or more standard deviations, or within ¬±10 or ¬±5 of the stated value. It is understood that, where a parameter range is provided, all integers within that range, and tenths thereof, are also provided. For instance, the range “0.1-80%” includes 0.1%, 0.2%, 0.3%, etc. up to and including 80%.

The terms “preferred” and “preferably” refer to embodiments of the invention that may afford certain benefits, under certain circumstances.

Unless otherwise stated, the term “room temperature” essentially means temperature in the range of 20-35° C.

The term “purity” means purity as determined by high-performance liquid chromatography (“HPLC”).

The term “compound of Formula II” refers to the compound shown above, and can be noted with the chemical name 3-(5-(difluoromethoxy)-1-methyl-3-(trifluoromethyl)-1H-pyrazol-4-yl)methylsulfanyl-4H-5,5-dimethylisoxazole.

The term “sulfoxide impurity” as used herein, refers to the compound of Formula III, as shown below.

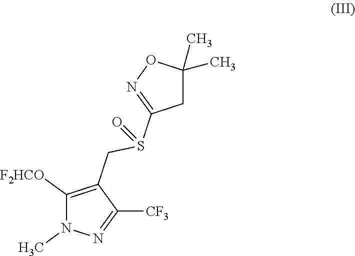

As used herein, the term “substantially free of sulfoxide compound of Formula III” refers to pyroxasulfone containing less than or equal to 1% of the sulfoxide compound of Formula III. Preferably, the term refers to pyroxasulfone containing less than 0.5% w/w of the sulfoxide compound of Formula III, more preferably less than 0.2% w/w, and further preferably less than 0.1%. In other instances, “substantially free of sulfoxide compound of Formula III” is less than 0.01%, 0.02%, or 0.05%.

The term “sulfonic acid impurity” as used herein, refers to the compound of Formula IV, as shown below.

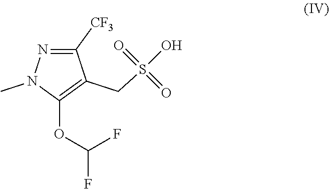 [SMILES: Cn1nc(C(F)(F)F)c(CS(=O)(=O)O)c1OC(F)F]

The term “continuous” or “continuous-flow” as used herein refers to a process in which one or more reagent streams flow from one reaction step to the next without an intervening isolation or purification step. In certain embodiments, one reaction step in the overall process of the claimed invention can be continuous-flow, while the other steps are batch-type steps.

The term “batch” as used herein refers to a process of manufacturing in which products are made in a discontinuous fashion using an array of processes.

The term “line” as used herein is not particularly limited and should be known to a person of skill in the art. In general, a line refers to, for example and without limitation, a tube, conduit, or pipe for conveying or transporting fluids. In a continuous flow process, the line can be designed to allow charging and/or discharging of fluids, such as reactants or products. In addition, the line (such as, in a reaction mixing line) can be designed to receive reactants and allow mixing and/or reaction of the reactants. Where the line is designed to receive reactants, the size and shape of the line can be adapted to enhance mixing and permit flow of the reactants into the line, minimizing back pressure.

The term “reactor” or “vessel” as used herein are not particularly limited and should be known to a person of skill in the art. In general, a reactor or vessel relates to, for example and without limitation, a container or vat designed to receive chemicals for a chemical process, such as a chemical reaction. In a continuous-flow process, the reactor or vessel can be designed to receive continuous charge of the reactants, followed by a continuous discharge of the products. Optionally, the reactants can remain in the reactor for a residence time to allow mixing and/or reaction of the reactants to form the products. The reactor or vessel can be provided with means, such as an agitator or baffles to allow mixing of the reactants.

The term “residence time” or “retention time” used herein refers to the time it takes for a molecule in a reagent stream to travel the entire length of a reactor. The residence time or retention time for a reagent stream in a reactor may depend on the length and width of the reactor as well as the flow rate of the reagent stream.

The continuous-flow process as described herein is not particularly limited and can be further modified by a person of ordinary skill in the art within the scope of the invention. In general, a continuous-flow process can allow a continuous flow of reactants that can be charged in an initial reactor, vessel, or line, allowing mixing or reaction of the reactants to form products. This is followed by continuous flow of the reaction medium from the reactor, vessel, or line. Thus, a continuous-flow process can be considered as a process wherein one or more reactants are charged or fed into a first reactor, vessel, or line, while one or more reaction products are simultaneously removed during part of the reaction process. A continuous-flow process can employ a single reaction step or multiple reaction steps to be performed, where each step independently of the other can be a chemical reaction, separation, or purification.

According to the present invention, an improved process is provided for the preparation of pyroxasulfone and salts thereof. The improved process, more specifically, provides for the preparation of pyroxasulfone by the oxidation of the sulfide compound of Formula II in a solvent that contains one or more glymes, preferably in the absence of a metal catalyst, preferably in the presence of a carboxylic acid, and optionally is performed using a continuous-flow process.

It has now been recognized that the present invention can, in certain embodiments, provide several important advantages and benefits over the prior art. In some embodiments, the present invention enables the synthesis of pyroxasulfone using a lower amount of oxidant, such as hydrogen peroxide. This can be an important advantage, particularly in the large-scale manufacture of pyroxasulfone. Using lower amounts of an oxidant, such as hydrogen peroxide, can also minimize unwanted side reactions or runaway exothermic reactions.

Another advantage of embodiments of the present invention is the absence of metal catalysts. In preferred embodiments, the reaction is performed without the need for a metal catalyst. Typical procedures used in large-scale manufacturing of pyroxasulfone utilize metal catalysts, which increases costs when scaled to the commercial level. The present invention allows the reaction to be performed at rates and efficiencies comparable to when the reaction is performed with a metal catalyst.

In certain embodiments, the reaction of making pyroxasulfone proceeds at a significantly and unexpectedly faster rate than prior art processes for making pyroxasulfone. This unexpected result of the present invention may lead to other advantages, particularly when manufacturing the product at large scale. For instance, manufacturing pyroxasulfone according to an embodiment of the present invention could be done at a lower cost due to the faster reaction time.

In certain embodiments, the synthesis of pyroxasulfone can be performed according to the present invention without the need to use toxic or unwanted solvents. For instance, the present invention enables one to manufacture pyroxasulfone without using toluene as a solvent. Toluene is a less-desired solvent, particularly for any product intended to be imported and/or sold into the United States because it avoids the use of less desirable solvents such as toluene due to toxicity.

Another advantage now recognized with embodiments of the present invention is that using glymes as a solvent improves the reaction work-up steps. The glyme solvent (or solvent systems) will be generally miscible with water. Therefore, the solvent will go with the mother liquor after the reaction and during extraction of the final product. Additionally, when a glyme is used as a solvent in accordance with the present invention, the water can be easily distilled off, leaving fully recovered glyme.

A further advantage of the present invention is the increased safety when performing the reaction at a commercial scale. An unexpected benefit of the present invention is that it is safer than current commercial manufacturing processes, minimizing the risk of run-away reactions or explosions. This is in part due to the usage of carboxylic acids which helps to stabilize the oxidant.

The improved process, more specifically, provides for the preparation of pyroxasulfone by the oxidation of the sulfide compound of Formula II in a solvent that contains one or more glymes, in the absence of a metal catalyst, in the presence of a carboxylic acid.

Another aspect of the invention is the preparation of pyroxasulfone by the oxidation of the sulfide compound of Formula II in a solvent that comprises one or more glymes, wherein the pyroxasulfone produced is substantially free of the sulfoxide compound of Formula III.

In accordance with the present invention, the process of making pyroxasulfone uses a solvent that comprises one or more glymes. In certain embodiments, the glyme can be selected from the group of glymes comprising monoglyme (1,2-dimethoxyethane), ethyl glyme, diglyme (diethylene glycol dimethyl ether), ethyl diglyme, triglyme, butyl diglyme, tetraglyme, and polyglymes. In certain preferred embodiments, the glyme is selected from the group consisting of a monoglyme (1,2-dimethoxyethane), diglyme (diethylene glycol dimethyl ether), and triglyme. In a more preferred embodiment, the glyme is monoglyme or diglyme. In a further preferred embodiment, the glyme is diglyme.

In a preferred embodiment, the solvent system used consists of or consists essentially of one or more glymes, and wherein the one or more glymes are as described above for various specific embodiments.

In certain embodiments, the solvent consists of or consists essentially of (a) one or more glymes and (b) one or more additional solvent components. Other additional solvents can include any solvents typically used. In preferred embodiments, the one or more additional solvent components is less then 10% of the solvent (whether by volume or weight). In other preferred embodiments, the one or more additional solvent components is less then 5% of the solvent (whether by volume or weight), less then 4% of the solvent (whether by volume or weight), less then 3% of the solvent (whether by volume or weight), less then 2% of the solvent (whether by volume or weight), or less then 1% of the solvent (whether by volume or weight). By way of an example, in one embodiment, the process according to the present invention is performed in a solvent system consisting of 95% diglyme and 5% toluene. In another embodiment, the process according to the present invention is performed in a solvent system comprising 2-3% acetic acid and 97-98% diglyme.

In an embodiment, the oxidizing agent used is selected from, but not limited to, an organic peroxide such as m-chloroperbenzoic acid, performic acid, peracetic acid and the like; or an inorganic peroxide such as hydrogen peroxide, potassium permanganate, sodium periodate and the like. In an embodiment, the oxidizing agent used is hydrogen peroxide.

Various forms of hydrogen peroxide can be used. The form of the hydrogen peroxide can be suitably selected by a person skilled in the art. In view of safety, danger, and economic efficiency considerations, a preferred form of the hydrogen peroxide includes a 10 wt % to 70 wt % aqueous hydrogen peroxide solution, more preferably a 15 wt % to 60 wt % aqueous hydrogen peroxide solution, still more preferably a 20 to 50 wt % aqueous hydrogen peroxide solution, and particularly preferably a 30 to 50 wt % aqueous hydrogen peroxide solution. Specific examples of the form of the hydrogen peroxide include, but are not limited to, a 25 wt % aqueous hydrogen peroxide solution, a 30 wt % aqueous hydrogen peroxide solution, a 35 wt % aqueous hydrogen peroxide solution and a 40 wt % aqueous hydrogen peroxide solution.

In some embodiments, the molar ratio of oxidizing agent to the compound of Formula II ranges from 1:1 to 5:1. In a more preferred embodiment, the molar ratio of oxidizing agent to a compound of Formula II ranges from 3:1 to 5:1. In a further preferred embodiment, the molar ratio of oxidizing agent to a compound of Formula II is 4:1.

In certain embodiments, the oxidizing agent is added slowly, e.g., across about 0.5 hours to about 4 hours, at temperature ranging from 25 to 95° C. In other embodiments of the invention, the oxidizing agent is adding more quickly, for example, in less than 0.5 hours, such as fifteen minutes. Depending on the overall amounts of reactants used, the oxidizing agent can be added at slower or faster rates.

In a preferred embodiment, the process of making pyroxasulfone as described herein is performed in the absence of a metal catalyst. Performing the oxidation reaction in the absence of a metal catalyst offers benefits such as easier work up steps and reduced costs.

In certain embodiments, the oxidation reaction can be performed in the presence of a non-metal catalyst. In a preferred embodiment, the non-metal catalyst used is a carboxylic acid. In a further preferred embodiment, the reaction is performed using a carboxylic acid that is acetic acid.

The oxidation step can optionally be performed in the presence of a metal catalyst. Examples of the metal catalyst include, but are not limited to, the following: tungsten catalysts (e.g., tungstic acid, tungstic acid salts (e.g., sodium tungstates (including sodium tungstate dihydrate and sodium tungstate decahydrate), potassium tungstate, calcium tungstate and ammonium tungstate), metal tungsten, tungsten oxides (e.g., tungsten(VI) oxide; tungsten(VI) oxide is also called tungsten trioxide), tungsten carbide, tungsten chlorides (e.g., tungsten(VI) chloride; tungsten(VI) chloride is also called tungsten hexachloride), tungsten bromides (e.g., tungsten(V) bromide), tungsten sulfides (e.g., tungsten(IV) sulfide; tungsten(IV) sulfide is also called tungsten disulfide), phosphotungstic acid and salts thereof (e.g., phosphotungstic acid, sodium phosphotungstate and ammonium phosphotungstate), silicotungstic acid and salts thereof (e.g., silicotungstic acid and sodium silicotungstate), and a mixture thereof), molybdenum catalysts (e.g., molybdic acid, molybdic acid salts, (e.g., sodium molybdate (including sodium molybdate dihydrate), potassium molybdate, ammonium molybdate (including ammonium molybdate tetrahydrate), metal molybdenum, molybdenum oxides (e.g., molybdenum(VI) oxide; molybdenum(VI) oxide is also called molybdenum trioxide), molybdate chlorides (molybdenum(V) chloride; molybdenum(V) chloride is also called molybdenum pentachloride), molybdenum sulfides (e.g., molybdenum(IV) sulfide; molybdenum(IV) sulfide is also called molybdenum disulfide), phosphomolybdic acid and salts thereof (e.g., phosphomolybdic acid, sodium phosphomolybdate and ammonium phosphomolybdate), silicomolybdic acid and salts thereof (e.g., silicomolybdic acid and sodium silicomolybdate), bis(2,4-pentandionato) molybdenum(VI) dioxide, and a mixture thereof), iron catalysts (e.g., iron(I) acetylacetoneate, iron(I) chloride and iron(I) nitrate, and a mixture thereof), manganese catalysts (e.g., potassium permanganate, manganese(II) oxide and manganese(II) chloride, and a mixture thereof), vanadium catalysts (e.g., vanadyl acetylacetonate, vanadium(V) oxide, vanadium(V) oxytrichloride, vanadium(V) oxytriethoxyde and vanadium(V) oxytriisopropoxide, and a mixture thereof), niobium catalysts (e.g., niobium carbide, niobium(V) chloride and niobium(V) pentaethoxyde, and a mixture thereof), tantalum catalysts (e.g., tantalum carbide (TaC), tantalum(V) chloride (TaCl₅) and tantalum(V) pentaethoxyde (Ta(OEt)₅), and a mixture thereof), titanium catalysts (e.g., titanium tetrachloride, titanium trichloride and titanium(IV) tetraisopropoxide, and a mixture thereof), zirconium catalysts (e.g., zirconium dioxide, zirconium(I) chloride, zirconium(IV) chloride, zirconium chloride oxide, and a mixture thereof), copper catalysts (e.g., copper(I) acetate, copper(II) acetate, copper(I) bromide and copper(I) iodide, and a mixture thereof), thallium catalysts (e.g., thallium(I) nitrate, thallium(I) acetate and thallium(I) trifluoroacetate, and a mixture thereof

In an embodiment, the metal catalyst used is a tungsten catalyst selected from tungsten, tungstic acid, tungstic acid salt, metallic tungsten, tungsten oxide, tungsten carbide or mixtures thereof. More preferably, the tungsten catalyst used is sodium tungstate or sodium tungstate dihydrate.

In an embodiment, the amount of the metal catalyst used is in the range of catalytic amount to 0.1 moles with respect to compound of Formula II. In a preferred embodiment, no metal catalyst is used.

The oxidation reaction according to the present invention can generally be performed at a temperature from about 25° C. to about 120° C. In preferred embodiments, the present invention will employ a temperature range of about 40° C. to about 115° C., of about 50° C. to about 110° C., of about 60° C. to about 105° C., of about 70° C. to about 100° C., or of about 75° C. to about 95° C. In further preferred embodiments, the present invention employs a temperature range of 90° C. to 95° C.

In certain embodiments, the present invention will provide for the preparation of pyroxasulfone that is faster than traditional or known methods. In certain embodiments, the process of making pyroxasulfone according to the present invention will be completed in 80% of the time as compared to an equivalent process that uses a non-glyme solvent. In other embodiments of the present invention, the process will be completed in 90% of the time, 70% of the time, 60% of the time, or 50% of the time as compared to an equivalent process that uses a non-glyme solvent.

In an embodiment, the process of the present invention comprises a step wherein the oxidation reaction is started at room temperature and then is progressed to a higher temperature ranging from about 40 to about 100° C. In a preferred embodiment, the process of the present invention involves the reaction starting at a temperature ranging from about 75° C. to about 95° C.

Yield: In an embodiment, pyroxasulfone is obtained in yield of more than 50%, preferably more than 70%. In further embodiments, the pyroxasulfone made according to the present invention has a yield of at least 80%, 85%, 90%, 95%, 97%, 98%, 99%, and 99.5%. In further preferred embodiments, the pyroxasulfone made according to the present invention has a yield of at least 95%.

Generally, the pyroxasulfone obtained according to the present invention will be obtained in at least 50% purity. In other embodiments, the product obtained will have a purity of about 60%, 70%, 80%, 90%, or 95% purity. In an embodiment, the present invention provides pyroxasulfone having purity of more than 95%, preferably more than 98%, further preferably over 99%. Other specific purity levels achievable through the present invention include 99.1%, 99.2%, 99.3%, 99.4%, 99.5%, 99.6%, 99.7%, 99.8%, 99.9%, 99.95%, and 99.99%.

In further embodiments, the process of the invention further comprises one or more purification steps, after the oxidation step described herein. The purification steps can include crystallization, chromatography, distillation, filtration, and the like. In an embodiment, the purification step used involves collecting the reaction mixture and quenching excess oxidizing agent, followed by washing with water and filtration. In another embodiment, the purification step used

In other embodiments, the present invention further comprises making a salt or solid-state form of pyroxasulfone based on the process disclosed herein. Different salts and solid- state forms (including solvated forms) of an active ingredient may possess different properties. Such variations in the properties of different salts and solid-state forms and solvates may provide a basis for improving formulation, for example, by facilitating better processing or handling characteristics, improving the dissolution profile, or improving stability (polymorph as well as chemical stability) and shelf-life. These variations in the properties of different salts and solid-state forms may also provide improvements to the final formulation or recipe, for instance, if they serve to improve dissolution. Different salts and solid-state forms and solvates of an active ingredient may also give rise to a variety of polymorphs or crystalline forms, which may in turn provide additional opportunities to use variations in the properties and characteristics of a solid active ingredient for providing an improved product.

A solid-state form (or polymorph) may be referred to herein as polymorphically pure or as substantially free of any other solid state (or polymorphic) forms. As used herein in this context, the expression “substantially free of any other forms” will be understood to mean that the solid-state form contains about 20% or less, about 10% or less, about 5% or less, about 2% or less, about 1% or less, or 0% of any other forms of the subject compound as measured, for example, by XRPD. Thus, the solid-state form of pyroxasulfone described herein as substantially free of any other solid-state forms would be understood to contain greater than about 80% (w/w), greater than about 90% (w/w), greater than about 95% (w/w), greater than about 98% (w/w), greater than about 99% (w/w), or 100% of the subject solid-state form of pyroxasulfone. Accordingly, in some embodiments of the disclosure, the described solid-state form of pyroxasulfone may contain from about 1% to about 20% (w/w), from about 5% to about 20% (w/w), or from about 5% to about 10% (w/w) of one or more other solid-state forms of the same pyroxasulfone.

By way of example, one embodiment of the process of the present invention further comprises preparing a crystalline form A of pyroxasulfone, as disclosed in WO2021144796A1, which is incorporated by reference in its entirety, in particular pages 4-8. The process comprises (a) converting the compound of Formula II into pyroxasulfone, according any one of the methods as described herein (e.g., using a glyme as a solvent and optionally under continuous-flow conditions); (b) about 100 mg of pyroxasulfone, prepared according to said methods, is taken in a 4 mL glass vial and ethyl acetate (1 mL) is added. The vial is shaken on an Eppendorf ThermoMixer C at 800 RPM/25° C. for 5 minutes, until the solution is clear by observation. The solution is slowly cooled to 0° C. and left standing for about 3 hours at 0° C. The obtained solid is removed from the solvent by decantation and dried at ambient condition. Solid was then ground with spatula and analyzed by XRD.

Crystalline Form A of pyroxasulfone that can be made according the present invention will be characterized by, for example, data selected from one or more of the following: (a) an XRPD pattern having peaks at 5.0, 10.0, 20.0, 25.1 and 30.3 degrees 2-theta±0.2 degrees 2-theta; (b) an XRPD pattern having peaks at 5.0, 10.0, 20.0, 25.1 and 30.3 degrees 2-theta±0.2 degrees 2-theta, and also having one, two or three additional peaks selected from 17.8, 20.9 and 22.4 degrees 2-theta±0.2 degrees 2-theta. Other embodiments include products made by any of the preferred embodiments described herein.

In other embodiments, the process of the present invention can be performed as a continuous-flow reaction. Typically, in a continuous-flow reaction, one or more reagents are pumped, optionally through a mixing junction, and then flow through a tube, conduit, or other vessel that can be heated, cooled, or subject to other conditions that facilitate the desired chemical reaction, such as irradiated with photons. The composition of the reaction is typically continuously monitored, such as by a light-absorbance detection method, or other methods described herein, while the reactant stream flows and mix continuously. In other embodiments, the reagents are pumped together at a mixing junction (e.g., through a mixer) and flow into a reactor chamber that can be heated, cooled, or subject to some other condition, to allow the reaction to proceed to completion.

A notable benefit of the continuous-flow process of making pyroxasulfone as described herein is the high modularity of the process of the present invention. By adding more reactors and lines to a continuous flow process, a manufacturer can expand the continuous flow process to not only cover one step of a reaction in greater volume, but all the prior steps required as well to create a single integrated process. This can be compared to traditional batch vessel synthesis, in which there is significant costs and difficulties involved in expanding the scope of production or attempting to integrate prior steps into part of the same process.

Utilizing continuous flow in the process of industrial production of pyroxasulfone has one or more benefits over traditional batch vessels such as the following: (i) mass and heat transfer can be significantly improved by decreasing reactor size; (ii) greater variation in the conditions that a reaction can be performed under, such as high pressures that would be unsafe or unfeasible to do in a batch vessel; (iii) greater safety due to the lower volumes involved in the reaction at any given time when performing highly energetic reactions; (iv) scalability of the reaction can be easily achieved via the usage of larger reactors or increasing the number of reactors; (v) greater control over variables such as heating and mixing.

The various embodiments of the process for making pyroxasulfone of the present invention offer numerous benefits over conventional and/or batch methods of making pyroxasulfone. For instance, the continuous-flow method of preparing pyroxasulfone, as described herein, offers and enables efficient and facile scaling of reaction volumes, making it advantageous for both R&D and large-scale industrial production. In other instances, the continuous-flow method of preparing pyroxasulfone, as described herein, reduces waste and conserves energy and resources, as compared to a comparative conventional and/or batch process for manufacturing pyroxasulfone. The various embodiments also enable manufacturing processes that are safer as compared to conventional and/or batch processes, such that there is a lower risk of accidents and/or environmental contamination. The continuous-flow processes of the present invention also enable certain manufacturing processes that are not feasible using a batch process.

An embodiment of the present invention involves a continuous-flow method in which a compound of Formula II dissolved in a solvent comprising one or more glymes, preferably diglyme, is introduced through the first feed line that is in fluid communication with the reactor unit. In a preferred embodiment, compound of Formula II is dissolved in a solvent containing diglyme and acetic acid. An oxidizing agent is then introduced through the second feed line that is in fluid communication with the reactor unit. The two reactants are then mixed within the reaction chamber, e.g., through flow diffusion or turbulent mixing. In a preferred embodiment, the oxidizing agent utilized is hydrogen peroxide at about 30% to about 50% strength.

In an embodiment of the present invention, there is a continuous-flow process for the preparation of pyroxasulfone comprising: (a) charging a compound of Formula II in a solvent comprising one or more glymes and a carboxylic acid through the first feed line that is in fluid communication with the reactor unit, in a continuous flow; (b) charging an oxidizing agent through a second feed line that is in fluid communication with the reactor unit, in a continuous flow; and (c) oxidizing a compound of Formula II with oxidizing agent in the reactor to form a product stream of pyroxasulfone.

According to an embodiment of the present invention, the flow rate of reactants in the first feed line is approximately 0.1 g/min to 10 g/min. In a preferred embodiment, the flow rate of reactants in the first feed line is 1 g/min to 8 g/min. In a more preferred embodiment, the flow rate of the reactants in the first feed line is 2 to 6 g/min. In a further preferred embodiment, the flow rate of the reactants in the first line is 3.0, 3.5, 4.0, 4.5, or 5.0 g/min.

According to an embodiment of the present invention, the flow rate of reactants in the second feed line is approximately 0.1 g/min to 5 g/min. In a preferred embodiment, the flow rate of reactants in the second feed line is 0.2 g/min to 2.5 g/min. In a more preferred embodiment, the flow rate of the reactants in the second feed line is 0.2 to 1.0 g/min. In a further preferred embodiment, the flow rate of the reactants in the second feed line is 0.2, 0.3, 0.4, 0.5. 0.6. 0.7, 0.8 or 0.9 g/min.

In certain embodiments of the continuous-flow process, the one or more feed lines used are made from PTFE (polytetrafluoroethylene) or PFA (perfluoroalkoxy). In certain embodiments, the outer diameter of the feed lines is ⅛″ or 1/16″ to best fit ¼-28 HPLC fittings. In certain embodiments, the inner diameter of the feed lines is 0.04″ or 0.02.″ In a preferred embodiment, the feed lines used are made from PTFE with an outer diameter of approximately ⅛ inch and an inner diameter of approximately 0.04 inch. Other variations may be permitted, depending on the particular reaction conditions and the specific needs to be in fluid communication with the continuous-flow reactors.

In certain embodiments, the continuous-flow reaction comprises one or more of the following arrangements: (a) reagents A and B are fed into the reactor compartment and flow continues through the reactor compartment, producing product C; (b) reagent A is fed into the reactor compartment in which reagent B is present as a solid-supported reagent, and flow continues through the reactor compartment, producing product C; (c) reagents A and B, together with a catalyst, are fed into the reactor compartment and flow continues through the reactor compartment, producing product C; and (d) reagents A and B are fed into the reactor compartment in which the catalyst is present as a solid-supported catalyst, and flow continues through the reactor compartment, producing product C. Other variations of the aforementioned embodiments are possible, for example, adding heat by through various means to the reaction compartment to facilitate the reaction.

In certain embodiments, the feed pumps that are used to drive the reactants through the feed lines comprise one of or any combination of HPLC pumps, metering pumps, gear pumps, syringe pumps, or peristaltic pumps. The feed pumps can be used to optimize reaction conditions, such that the final product is optimized, e.g., increased purity and/or increased yield. Within the scope of the present invention is a process that uses multiple feed pumps to drive the various reactants through the reactor system. For instance, for the multistep processes described herein, the process may include a separate feed pump for each reactant that is added to the continuous-flow system. For example, in certain embodiments wherein the compound of Formula II is being produced through a continuous-flow process, one feed pump is used to pump the compound of Formula II through one line into the reactor while a second feed pump is used to pump oxidizing agent through another line into the reactor.

In certain embodiments, pressure and similarly temperature are controlled in the reactor by back-pressure regulators, which are typically spring-loaded valves that maintain a constant pressure within the reactor itself. Back-pressure regulators come in various configurations, including self-operated, high-flow, differential, vacuum, air-loaded, spring-loaded, diaphragm-based and pilot-operated. In certain embodiments, the pressure differential can be in approximately the range of from 1 bar to about 9 bar. In other embodiments, the pressure differential can be approximately 1 bar, 2 bar, 3 bar, 4 bar, 5 bar, 6 bar, 7 bar, 8 bar, or 9 bar.

In certain embodiments of the present invention, the reactor used can be selected from three main types of reactors for a continuous-flow process of the present invention: (1) continuous-stirred tank reactors; (2) tubular reactors, and (3) packed-bed reactors. In a continuous-stirred tank reactor, there is generally a single large tank that has entrances and exits for reactants and products, with the reaction occurring within said tank while being mixed through active stirring. In a tubular reactor, the reaction takes place within a cylindrical pipe, where the reactants flow in and out. Tubular reactors can also take the form of coiled tubular reactors (coil reactors), in which the reaction takes place within a coil of tubing, with mixing of reactants frequently being through diffusion. In a packed-bed reactor, some form of solid, e.g., a catalyst or substrate, is typically fixed onto the bed of the reactor, over which a liquid reactant will then flow over. In a preferred embodiment of the present invention, the type of reactor used is a tubular reactor.

In certain embodiments of the present invention, the reactor used can be a tube-in-tube reactor for a continuous-flow process of the present invention. A tube-in-tube reactor is a reactor comprising a gas-permeable inner tube encased within a non-permeable outer tube and is particularly useful for gas-liquid reactions. A tube-in-tube reactor may be based on the Teflon AF membrane. Two additional configurations of the tube-in-tube reactor use the polytetrafluoroethylene material or the stainless-steel outer tubes. In certain embodiments in which the oxidizing agent used takes the form of a gas (such as chlorine, oxygen, or ozone), a tube-in-tube reactor can be used to oxidize the compound of Formula II to form pyroxasulfone. Where appropriate, a tube-in-tube reactor can be employed in other steps of the multi-step continuous-flow process for making pyroxasulfone, as described herein.

In other embodiments, the reactor is made from materials such as perfluorinated polymers, stainless steel, glass, ceramics, silicon glass, and combinations thereof. In a preferred embodiment, the reactor is made from stainless steel, which may be optionally lined.

In certain embodiments using a tubular reactor, the inner diameter of the tubular reactor is between 1 inch to 4 inches, and the outer diameter of the tubular reactor is between 1.5 inches to 5 inches. Other suitable parameters for the inner diameter of the tubular reactor include 1.0, 1.1, 1.2, 1.3, 1.4, 1.5, 1.6, 1.7, 1.8, 1.9 or 2.0 inches. Other suitable parameters for the outer diameter of the tubular reactor include between 1.5, 1.6, 1.7, 1.8, 1.9, 2.0, 2.1, 2.2, 2.3, 2.4, 2.5, 2.6, 2.7, 2.8, 2.9 or 3.0 inches.

In some embodiments, the continuous-flow reactor system used can be smaller scale to employ micro-or meso-fluidic reactors, which are useful for synthesizing compounds on the microgram to kilogram level respectively. These miniature reactor systems allow for greater extremes in heat and pressure while minimizing risk due to the smaller volumes involved. In many instances, the microreactor systems and processes offer greater efficiencies comparted to conventional batch-process chemistry for making pyroxasulfone.

In some embodiments, the continuous-flow reactor system itself can be further split into functional parallel micro-reaction systems to further increase production capacity. Said micro-reaction systems typically comprise one or more mixing reactors, one or more micro-reactors (such as a micro-channel reactor), and one or more heating or cooling elements or any combinations thereof that might be jacketed to maintain the desired pressure or temperature of the reaction vessels in the system. In certain arrangements, the fully continuous-flow micro-reaction process utilizes a system comprising a first micro-channel reactor, a first micro-mixer, a second micro-channel reactor, a second micro-mixer and an extraction-separation unit communicated in sequence; the extraction-separation unit being composed of at least one extraction separator. In preferred embodiments, a micro-reactor system is used with continuous-flow step of oxidizing the compound of Formula II with hydrogen peroxide to form pyroxasulfone, as described herein (e.g., using a glyme as a solvent, preferably in the absence of a metal catalyst, preferably in the presence of a carboxylic acid (such as acetic acid, 0.1 equivalents). In some of these preferred embodiments, the micro-channel reactor is a tubular micro-channel reactor, a plate-type micro-channel reactor, or other commercially available micro-channel reactors.

In certain embodiments, the reagents in the reactor are mixed using diffusion, a static mixer, a coaxial flow mixer, or a continuous stirred-tank reactor. In a preferred embodiment, the reagents in the reactor are mixed using diffusion or a static mixer. A diffusion mixer will generally maintain laminar flow conditions to promote gentle mixing. One or more diffusion mixers can be implemented in the process of the present invention by extending the tube length or dividing the flow into multiple laminates, thereby increasing the interfacial area between fluidic layers. In other embodiments, the process will utilize convective diffusion enhancement. This technique involves inducing a turbulent flow regime to deform the lamellar arrangements, resulting in more thorough mixing. As noted, in other embodiments, static mixers may be used. Static mixers are preferred in large-scale reactions of the present invention. Static mixers may use tube inserts, such as blades/helices or fluid stretching, to generate turbulence and intense radial mixing. Of course, reactor solutions can be mixed prior to entry into the continuous-flow reactor via the one or more feed lines.

By employing a range of mixer designs and techniques, the continuous-flow process of the present invention enables precise control and optimization of the mixing process, contributing to improved reaction outcomes for preparing pyroxasulfone.

In certain embodiments, the reaction process can be coupled with the use of ultrasound. Ultrasound can accelerate reaction rates or act as a catalyst for reactions. The use of ultrasound also improves the mixing of reagents for faster and more efficient reactions.

In some instances, mass flow controllers may be used to facilitate the introduction of gases into a liquid line. Mass flow controllers are designed to regulate and control the amount and flow rate of gases in the system. In certain embodiments wherein the oxidizing agent is a gas, or a step toa multi-step continuous-flow process for making pyroxasulfone.

In certain embodiments, the reactor is heated using water baths, hot steam, electric heating, boiler heating, or microwave heating. In preferred embodiments, the reactor is heated using water baths or electric heating.

The continuous-flow process for making pyroxasulfone is suitable for being conducted at various scales. In preferred embodiments, the process is tailored to manufacture pyroxasulfone at commercial scales, ranging from 0.1 kg to 1000 kg. According to an embodiment of the present invention, the volume of the reactors for carrying out the continuous-flow process of oxidizing the compound of Formula II to produce pyroxasulfone at a commercial scale are selected from various capacities ranging from 1 L, 10 L, 50 L, 100 L, and more, which can be based on the desired output of pyroxasulfone.

One advantage of the continuous-flow process of the present invention is that it can be performed with greater efficiency with respect to time compared to batch processes. According to an embodiment, the preparation of pyroxasulfone through continuous-flow occurs in a shorter reaction time, relative to known methods used at an industrial scale. For instance, in certain embodiments, the preparation of pyroxasulfone can be completed in 90% of the time, 80% of the time, 70% of the time, 60% of the time, or 50% of the time as compared to a comparative or conventional batch process. In a preferred embodiment, the preparation of pyroxasulfone occurs in 50% of the time as compared to a comparative or conventional batch process using known methods.

In other embodiments, the reaction chamber of the tubular reactor is preheated to a temperature between 50° C. to 100° C. before the reaction components are introduced into the chamber. In a preferred embodiment, the reaction chamber of the tubular reactor is preheated to 95° C. The reaction chamber is then gradually modulated to a temperature between 80° C. and 100° C. as reactants are added. In a preferred embodiment, the reaction chamber is modulated to a temperature between 90° C. to 95° C. as the reactants are added.

In another embodiment, the continuous-flow reaction is heated using inductive heating. Inductive heating is a heating technology based on the induction of an electromagnetic field (at medium or high frequency depending on nanoparticle sizes) to magnetic nanoparticles within the reaction chamber. The induction results in a very rapid increase of temperature, which allows for greater control over the reaction temperature. Additionally, induction heating does not require any encasement of reaction apparatuses for safety purposes. The process is also highly efficient, as induction heating has high power-transfer value.

In another embodiment, one or more steps in the continuous-flow reaction is heated using microwave heating. Microwave heating utilizes microwaves to heat the fluids present in a reaction medium. This method of heating offers the benefits of being able to rapidly reach target temperatures as well as being energy efficient. Of course, microwave heating may not be appropriate for all chemical reactions performed in the continuous-flow process of making pyroxasulfone, which can be determined by a person of ordinary skill in the art.

In an embodiment, the residence time of the reactants for the oxidation step in the continuous-flow reactor is typically anywhere between 1 to 6 hours. In a preferred embodiment, the residence time of the reactants in the reactor is 2.0, 2.5, 3.0, 3.5, or 4.0 hours.

In an embodiment, the oxidation of pyroxasulfone occurs as depicted below. In said embodiment, compound of Formula II and hydrogen peroxide are pumped into a tubular reactor that is heated to 90° C. to 95° C. The reactants remain in the tubular reactor for a retention time of 3 hours, with the result of the reaction being pyroxasulfone.

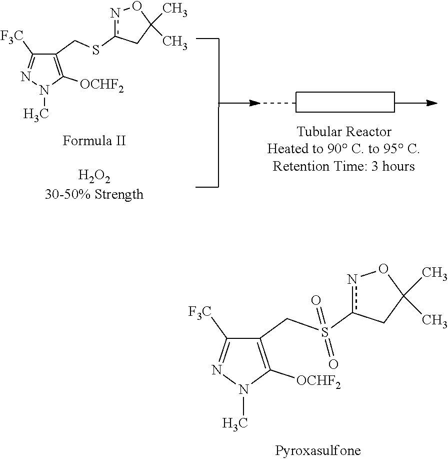

Particularly preferred embodiments employ a continuous-flow process for the preparation of pyroxasulfone (Formula I) or a salt thereof comprising: (a) preparing a first solution comprising a compound of Formula II and a carboxylic acid in a catalytic amount, wherein the first solution comprises one or more glymes as a solvent; (b) preparing a second solution comprising an oxidizing agent; (c) charging said first solution through a first feed line into a continuous-flow reactor unit, wherein the first feed line is fluidically connected with the continuous-flow reactor unit; (d) charging said second solution through a second feed line into the continuous-flow reactor, wherein the second feed line is fluidically connected with the continuous-flow reactor unit; (e) activating conditions in the continuous-flow reactor such that the first solution and the second solution interact, thereby effecting the oxidation of the compound of Formula II into pyroxasulfone, wherein said effecting the oxidation occurs preferably in the absence of a catalyst; and (f) optionally receiving the solution containing pyroxasulfone and then isolating the pyroxasulfone in solid form.

In these particularly preferred embodiments, certain processes are performed such that the glyme is diglyme; the carboxylic acid is acetic acid; the oxidizing agent is hydrogen peroxide at a concentration of 30% to 50%; the reaction is performed in the absence of a metal catalyst; the continuous-flow reactor unit is heated so that the reaction stream within the reactor unit is heated to about 80° C. to about 100° C., preferably 90° C. to 95° C.; the residence time is from about four to about six hours, preferably between about two hours and about four hours; the reactor is a tubular reactor or coil reactor; the pressure in the reactor is from about 1 bar to about 10 bar; and the purity of pyroxasulfone produced is greater than 99%, as measured by HPLC.

In these instances, a carboxylic acid in a catalytic amount can be a carboxylic acid in an amount that is equivalent to about 0.05, 0.07, 0.09, 0.1, and 0.2 equivalents of the compound of Formula II.

Another embodiment for the novel process for producing pyroxasulfone by continuous- flow process comprises: (a) charging first solution comprising a compound of Formula II, a carboxylic acid, and a suitable solvents through a first feed line into a continuous-flow reactor unit, in a continuous flow; (b) charging a second solution comprising an oxidizing agent and a suitable solvent through a second feed line into a reactor unit, in a continuous flow; and (c) oxidizing the compound of Formula II with the oxidizing agent in said reactor to form a reaction stream comprising pyroxasulfone, wherein said oxidizing is performed at a temperature of about 85° C. to about 100° C. and wherein the residence time in the continuous-flow reactor unit is from about 2 hours to about 5 hours. In this embodiment, the continuous-flow reactor unit is preferably a tubular or coil reactor. In this embodiment, the process can further comprise the addition of a metal catalyst selected from any of the catalysts described herein, preferably a metal catalyst such as a tungsten catalyst, a molybdenum catalyst, a titanium catalyst, a zirconium catalyst, and mixtures thereof. In this embodiment, the metal catalyst can be sodium tungstate. The metal catalyst can added as a separate solution to the reactor unit, or it can be included within either the first solution charging first solution comprising a compound of Formula II or the second solution comprising the oxidizing agent. In other instances, this continuous-flow embodiment uses a solvent other than a glyme.

As will be understood, the oxidation step to make pyroxasulfone (along with other steps for the multi-step processes described herein) can be performed in an inert atmosphere or conditions. Such conditions will preferably use nitrogen or argon gas to minimize the amount of reactive oxygen present in the reaction environment.

In a continuous-flow process for making pyroxasulfone, the product obtained from the continuous-flow process can be purified by various methods. The resulting reaction stream is collected in a suitable receiving vessel after the last continuous-flow reactor for the oxidation step. Suitable purification methods include washes with water combined with or without non-polar organic solvents such as heptane, hexane, or petroleum ether followed by drying. Other methods include filtration followed by washing with water and petroleum ether then drying. In an embodiment, the product is filtered and then washed with water and petroleum ether before drying. In a preferred embodiment, the product pyroxasulfone is quenched with the addition of sodium sulfite, then purified by washing with water followed by filtration. In a further preferred embodiment, the reaction stream obtained (containing the product pyroxasulfone) is quenched with the addition of sodium sulfite, then purified by washing with water and cooling to 0° C. followed by filtration and drying.

As one example, the reaction stream containing the pyroxasulfone formed according to the present process is collected in a suitable vessel. A 20% aqueous sodium sulfite solution is added to the reaction mixture, and the resulting mixture is stirred at an internal temperature of about 60° C. to 70° C. for thirty minutes. The mixture thus obtained is separated into an organic and aqueous layer. To the organic layer is added water, and the mixture is concentrated under reduced pressure. To the obtained crude product is added isopropanol, and crystals are separated by filtration at room temperature. The obtained crystals of pyroxasulfone are washed with additional isopropanol and water. The obtained crystals of pyroxasulfone are then dried using conventional means.

In other embodiments, the process of preparing pyroxasulfone under the continuous-flow process will include in-line extraction techniques to further increase the purity and reaction yield. In-line extraction is an extraction technique in which a liquid-liquid extraction is performed as part of a continuous-flow process by pumping a solvent with different affinity for the target compound into the reaction stream. In-line extraction can include, in certain embodiments, counter-current extraction (using a counter-current flow to separate desired products from impurities); in-line flash chromatography (integrating a flash chromatography system directly into the flow reactor to purify products; scavenger columns (using columns to remove unwanted byproducts or impurities); distillation (integrating distillation units to separate components based on boiling points; and nanofiltration (using membranes to separate molecules based on size). Depending on the particular reaction steps in the continuous-flow process, the in-line extraction can be employed at one or junctures between the multiple continuous-flow reactors.

In various embodiments of the continuous-flow process, the process employs one or more membrane separators that can remove certain waste reactants, such as for example inorganic waste or byproducts. Certain membrane separators are selectively permeable membranes useful in isolating volatile organic compounds. One or more membrane separators can be used at various points in the multi-step continuous-flow process for making pyroxasulfone. The one or more membrane separators would typically be used between reaction steps and, for example, between continuous-flow reactors. Additionally, the reaction stream containing crude pyroxasulfone after the final oxidation step can be filtered using a membrane separator to remove unwanted compounds from the reaction stream to purify pyroxasulfone.

In various embodiments, the process for preparing pyroxasulfone, particularly when performed as a continuous-flow process, can employ scavenger resins. Scavenger resins are polymers with functional groups attached that can react with specific compounds. Polymer-supported scavengers are used to remove an unwanted compound from a solution and/or reaction stream, and their use provides the advantages of solution-phase reactions. Specific polymer-supported scavengers can be employed between different stages of the continuous-flow process. Additionally, the purification of pyroxasulfone can be performed by passing the reaction stream after the last stem containing crude pyroxasulfone through scavenger resins with functional groups that react to a sulfonic acid impurity of Formula IV, such that the amount of the impurity is reduced in the final pyroxasulfone product.

In an embodiment, the continuous-flow process for preparing pyroxasulfone comprises steps to recycle the solvent used in the oxidation reaction as well as other reactions described in multi-step embodiments. In certain embodiments, once the purified pyroxasulfone has been isolated from the reaction stream, the remaining reaction medium comprising, for example, a glyme solvent, excess hydrogen peroxide, and aqueous waste can be treated such that the glyme solvent is isolated and re-used in subsequent processes. In an embodiment, the reaction medium after the oxidation step is treated by quenching the excess hydrogen peroxide with sodium bisulfite and distilling off remaining water such that diglyme is isolated for use in subsequent reaction.

In an embodiment, the reaction stream of crude pyroxasulfone can be purified through the use of HPLC columns. An HPLC column can be used to purify pyroxasulfone by pumping a solution containing pyroxasulfone through a column filled with adsorbent that has different affinities for pyroxasulfone, the sulfoxide impurity of Formula III, and the sulfonic acid impurity of Formula IV. By pumping the crude mixture through the HPLC column the components of the solution will separate due to their differing affinities, allowing for isolation of pyroxasulfone alone. In an embodiment, purification of pyroxasulfone through HPLC columns is performed as part of a continuous-flow process wherein the reaction stream of crude pyroxasulfone is passed through an HPLC column once it exits the continuous-flow reactor.

In the process of making pyroxasulfone through a continuous-flow method according to the present invention, reaction progress can be monitored through various forms of instrumentation including both inline sensors and atline/offline methods. In certain embodiments the inline sensors used measure temperature, pH, or UV absorption. In certain embodiments the atline or offline methods used include HPLC or mass-spectrometry. In a preferred embodiment, a combination of inline sensors that measure temperature and atline/offline methods involving HPLC are utilized.

In one embodiment of the present invention, the continuous-flow reaction progress is monitored, either automatically or manually, for sulfoxide impurities of Formula III and/or sulfonic acid impurities of Formula IV using inline sensors and/or atline/offline methods. The monitoring method used can comprise atline and/or offline analysis methods to detect the sulfoxide impurities of Formula III or sulfonic acid impurities of Formula IV are HPLC or mass-spectrometry. In a preferred embodiment, the continuous-flow process uses the atline/offline analysis method to detect the sulfoxide impurities of Formula III or sulfonic acid impurities of Formula IV is HPLC.

In other embodiments of the present invention, the resulting pyroxasulfone produced according to the novel process has a purity greater than 95% via HPLC analysis. In another embodiment, the resulting pyroxasulfone has a purity greater than 98% via HPLC analysis. In a preferred embodiment, the resulting pyroxasulfone has a purity greater than 99%, with undetectable amounts of the sulfide precursor and the sulfoxide intermediate. Undetectable amounts are, in certain embodiments, any amounts below 0.3 ppm, 0.2 ppm, or 0.1 ppm.

In an embodiment, samples can be taken for atline or offline analysis during the continuous-flow method through the usage of sample loops. In a sample loop, samples will flow into a six-way valve that can be switched between positions such that in one position the sample will flow into the sample loop that can then be isolated and analyzed, and a second position in which the sample will flow through to vent/waste for downstream collection or disposal.

In certain embodiments, the continuous-flow process comprises one or more back-pressure regulators (“BPR”). In a continuous-flow process according to the present invention, a BPR is used to control the pressure within the reactor, ensuring optimal reaction conditions and stability. The one or more BPRs maintain a constant pressure downstream, allowing for precise control of parameters such as temperature and flow rate. In certain embodiments, the BPR is installed at the reactor's outlet to maintain the specified pressure. Maintaining the reaction conditions within a specified pressure range helps control reaction rates, prevents boiling of solvents, keeps gases in solution, and otherwise aids in performing the reaction. In certain embodiments, an equilibar BPR is used (which is known for its simple design, wide flow range, and ability to handle a variety of chemistries and temperatures), or a heated BPR is used (which is designed to maintain elevated temperatures through the BPR.

In other embodiments of the present invention, the process of making pyroxasulfone comprises a multistep process for synthesizing pyroxasulfone, whereby the starting reactants can be selected from one or two (or more) of several components and then the last step of the multi-step process comprises the preparation of pyroxasulfone (Formula I) by the oxidation of the sulfide compound of Formula II in a solvent that comprises one or more glymes and that is preferably performed under continuous-flow conditions, as described herein.

In one multi-step embodiment, the process of producing pyroxasulfone begins with (a) the process of reacting (1-methyl-3-(trifluoromethyl)-1H-pyrazol-5-ol) with formaldehyde in presence of a base to obtain a compound of 4-(hydroxymethyl)-1-methyl-3-(trifluoromethyl)-1H-pyrazol-5-olate or salt thereof; (b) this is then followed by reacting 4-(hydroxymethyl)-1-methyl-3-(trifluoromethyl)-1H-pyrazol-5-olate or salt thereof with chlorodifluoromethane in a suitable solvent in presence of a base to obtain a compound of 5-(difluoromethoxy)-1-methyl-3-(trifluoromethyl)-1H-pyrazol-4-yl)methanol; (c) 5-(difluoromethoxy)-1-methyl-3-(trifluoromethyl)-1H-pyrazol-4-yl) methanol is then reacted with a chlorinating agent to obtain a compound of 3-(((5-(difluoromethoxy)-1-methyl-3-trifluoromethyl)-1H-pyrazol-4-yl)methyl)thio)-5,5-dimethyl-4,5-dihydroisoxazole; (d) 3-(((5-(difluoromethoxy)-1-methyl-3-trifluoromethyl)-1H-pyrazol-4-yl)methyl)thio)-5,5-dimethyl-4,5-dihydroisoxaz-ole is then reacted with 5,5-dimethyl-4,5-dihydroisoxazol-3-yl-carbamimidothioate to obtain a compound of Formula II, as described in WO 2020/240392, which is hereby incorporated by reference in its entirety and in particular pages 6-9; and (e) the compound of Formula II is then reacted with an oxidizing agent to obtain the compound of pyroxasulfone or salt thereof, as described herein (e.g., using a glyme as a solvent), preferably in the absence of a metal catalyst, preferably in the presence of a carboxylic acid (such as acetic acid, 0.1 equivalents) and optionally under continuous-flow conditions). In this embodiment, the entire process can be performed under continuous-flow conditions, batch conditions, or a combination thereof, and preferably with the last oxidation step being performed under continuous-flow conditions.

In another multi-step embodiment according to the present invention, the preparation of pyroxasulfone begins by reacting 5-hydroxy-1-methyl-3-(trifluoromethyl) pyrazole in a solution of NaOH with N,N′-dimethylcarbamimidothioic acid. A solution of N,N′-dimethylcarbamimidothioic acid, 4,5-dihydro-5,5-dimethyl-3-isoxazolyl ester hydrochloride is added to synthesize the intermediate 4-(((4,5-dihydro-5,5-dimethyl-3-isoxazolyl)thio)methyl)-1-methyl-3-(trifluoromethyl)-1H-pyrazol-5-ol. This intermediate is then reacted with a fluorinated methyl halide of formula F₂HC-L₁, where L₁ is a leaving group as described in WO2021/176456, which is hereby incorporated by reference in its entirety and in particular pages 6-11 and 23-24. This step is then followed by oxidation to form the compound of pyroxasulfone as described as described herein (e.g., using a glyme as a solvent preferably in the absence of metal catalyst, preferably in the presence of a carboxylic acid, and optionally under continuous-flow conditions).

In another multi-step embodiment according to the present invention, the preparation of pyroxasulfone begins by (a) reacting 5-hydroxy-1-methyl-3-(trifluoromethyl) pyrazole in a solution of NaOH with formaldehyde; (b) adding N,N-dimethyl(5,5-dimethyl-4H-isoxazol-3-ylsulfanyl)methaniminium bromide to synthesize 4-(((4,5-dihydro-5,5-dimethyl-3-isoxazolyl)thio)methyl)-1-methyl-3-(trifluoromethyl)-1H-pyrazol-5-ol; (c) reacting 4-(((4,5-dihydro-5,5-dimethyl-3-isoxazolyl)thio)methyl)-1-methyl-3-(trifluoro-methyl)-1H-pyrazol-5-ol with a fluorinated methyl halide of formula F₂HC-L₁, where L₁ is a leaving group as described in WO2021/176456, which is hereby incorporated by reference in its entirety and in particular pages 6-11 and 24. This step is then followed by oxidation to form the compound of pyroxasulfone as described as described herein (e.g., using a glyme as a solvent, preferably in the absence of a metal catalyst, preferably in the presence of a carboxylic acid (such as acetic acid, 0.1 equivalents), and optionally under continuous-flow conditions).

In another multi-step embodiment of the present invention, a method of making the compound of Formula II can be combined with the process disclosed herein, thereby providing a new and useful process for making pyroxasulfone that provides advantages over known processes. For instance, in certain embodiments, the novel process comprises (a) preparing a compound of Formula II according to the method as described in WO2023/194957, which is hereby incorporated by reference in its entirety and in particular pages 4-8, and (b) then converting the compound of Formula II into pyroxasulfone, as described herein (e.g., using a glyme as a solvent, preferably in the absence of a metal catalyst, preferably in the presence of a carboxylic acid (such as acetic acid, 0.1 equivalents), and optionally under continuous-flow conditions).

In another multi-step embodiment, the present invention comprises (a) reacting 3-ethanesulfonyl-5,5-dimethyl-2-isoxazoline with 2-(5-difluoromethoxy-1-methyl-3-trifluoromethyl-1H-pyrazole-4-yl-methyl)-isothiourea hydrobromide, which produces a compound of Formula II, and (b) then oxidizing the compound of Formula II as described herein to yield pyroxasulfone, as described herein (e.g., using a glyme as a solvent, preferably in the absence of a metal catalyst, preferably in the presence of a carboxylic acid (such as acetic acid, 0.1 equivalents), and optionally under continuous-flow conditions). The process of making the compound of Formula II can use a base in solvent, such as anhydrous potassium carbonate base in the presence of ethanol, water, and N,N-dimethyl formamide (DMF) solvent, as described in U.S. Pat. No. 7,256,298, which is hereby incorporated by reference in its entirety and in particular columns 30-33. In this embodiment, the entire process can be performed under continuous-flow conditions, batch conditions, or a combination thereof, and preferably with the last oxidation step being performed under continuous-flow conditions.

In another multi-step embodiment, the present invention comprises the thiomethylation of 1-methyl-3-(trifluoromethyl)-1H-pyrazol-5-ol through a reaction with potassium thioacetate to form thioacetic acid S-(5-hydroxy-1-methyl-3-trifluoromethyl-1H-pyrazol-4-ylmethyl) ester. The ester is then reacted with chlorodifluoromethane to form thioacetic acid S-(5-difluoromethoxy-1-methyl-3-trifluoromethyl-1H-pyrazol-4-ylmethyl) ester. This is then reacted with 3-chloro-5, 5-dimethyl-4,5-dihydroisoxazole to form a compound of Formula II as described in WO2021/019537, which is hereby incorporated by reference in its entirety and in particular pages 19 and 21-24. The compound of Formula II is then oxidized to form pyroxasulfone, as described herein (e.g., using a glyme as a solvent, preferably in the absence of a metal catalyst, preferably in the presence of a carboxylic acid (such as acetic acid, 0.1 equivalents), and optionally under continuous-flow conditions). In this embodiment, the entire process can be performed under continuous-flow conditions, batch conditions, or a combination thereof, and preferably with the last oxidation step being performed under continuous-flow conditions.

In another multi-step embodiment, 5-(difluoromethoxy)-1,4-dimethyl-3-(trifluoromethyl)-1H-pyrazole is reacted with a brominating agent to form 4-(bromomethyl)-5-(difluoromethoxy)-1-methyl-3-(trifluoromethyl)-1H-pyrazole, which is then reacted with 3-bromo-5,5-dimethyl-4H-isoxazole in potassium carbonate to form the compound of Formula II as described in WO2022/009044 and in particular pages 7-17, 20, and 22. The compound of Formula II is then oxidized to form pyroxasulfone, as described herein (e.g., using a glyme as a solvent and optionally under continuous-flow conditions). In this embodiment, the entire process can be performed under continuous-flow conditions, batch conditions, or a combination thereof, and preferably with the last oxidation step being performed under continuous-flow conditions.

In another multi-step embodiment, (5,5-dimethyl (4,5-dihydroisoxazolo-3-yl))thiocarboxamidine hydrobromide is reacted with sodium hydroxide and 4-chloromethyl-5-difluoromethoxy-1-methyl-3-trifluoromethyl-pyrazole to form a compound of Formula II as described in US20230012374, which is hereby incorporated by reference in its entirety and in particular pages 4-9 and 63. The compound of Formula II is then oxidized with hydrogen peroxide to form pyroxasulfone, as described herein (e.g., using a glyme as a solvent, preferably in the absence of a metal catalyst, preferably in the presence of a carboxylic acid (such as acetic acid, 0.1 equivalents), and optionally under continuous-flow conditions).

In another multi-step embodiment, 4-(chloromethyl)-5-fluoro-1-methyl-3-(trifluoromethyl)-1H-pyrazole is reacted with thiourea to form [5-Fluoro-1-methyl-3-(trifluoromethyl)-1H-pyrazol-4-yl]methyl carbamimidothioate hydrochloride. [5-Fluoro-1-methyl-3-(trifluoromethyl)-1H-pyrazol-4-yl]methyl carbamimidothioate hydrochloride is then reacted with 3-({[5-fluoro-1-methyl-3-(trifluoromethyl)-1H-pyrazol-4-yl]methyl}sulfanyl)-5,5-dimethyl-4,5-dihydro-1,2-oxazole to form 4-{[(5,5-dimethyl-4,5-dihydro-1,2-oxazol-3-yl) sulfanyl]methyl}-1-methyl-3-(trifluoromethyl)-1H-pyrazol-5-ol. 4-{[(5,5-dimethyl-4,5-dihydro-1,2-oxazol-3-yl)sulfanyl]methyl}-1-methyl-3-(trifluoro-methyl)-1H-pyrazol-5-ol is then reacted with difluorochloromethane in the presence of a base and an organic solvent to form a compound of Formula II as described in US20240279212, which is hereby incorporated by reference in its entirety and in particular pages 3-13 and 15-16. The compound of Formula II is then oxidized with hydrogen peroxide to form pyroxasulfone, as described herein (e.g., using a glyme as a solvent, preferably in the absence of a metal catalyst, preferably in the presence of a carboxylic acid (such as acetic acid, 0.1 equivalents), and optionally under continuous-flow conditions).

In another multi-step embodiment, 1,2-bis[(5-difluoromethoxy-1-methyl-3-trifluoromethylpyrazol-4-yl)methyl] disulfide is dissolved in dimethylformamide. 3-chloro-5,5-dimethyl-4,5-dihydroisooxazole, potassium carbonate and a Rongalit aqueous solution are then added to the solution of 1,2-bis[(5-difluoromethoxy-1-methyl-3-trifluoromethylpyrazol-4-yl)methyl] disulfide to form a compound of formula II as described in US20240391909, which is hereby incorporated by reference in its entirety and in particular pages 7-21. The compound of Formula II is then oxidized with hydrogen peroxide to form pyroxasulfone, as described herein (e.g., using a glyme as a solvent, preferably in the absence of a metal catalyst, preferably in the presence of a carboxylic acid (such as acetic acid, 0.1 equivalents), and optionally under continuous-flow conditions).

In certain embodiments wherein the continuous-flow process utilizes a multi-step process, one or more solvent-exchange steps may be employed. A solvent exchange involves the process of removing a first solvent of the reaction stream through, for example distillation, and then adding a second solvent to the reaction stream, thereby replacing the first solvent with the second solvent. Solvent exchange can be employed at one or more appropriate junctures in the multi-step continuous-flow process. For instance, in one embodiment, the present invention will comprise two reaction steps, with the second step being the oxidation step to form pyroxasulfone in a glyme solvent. The preceding step in this instance would employ a solvent that is not a glyme, and therefore a solvent exchange would be employed such that the last step, the oxidation step, is performed in a glyme solvent, along with any additional suitable reactants.

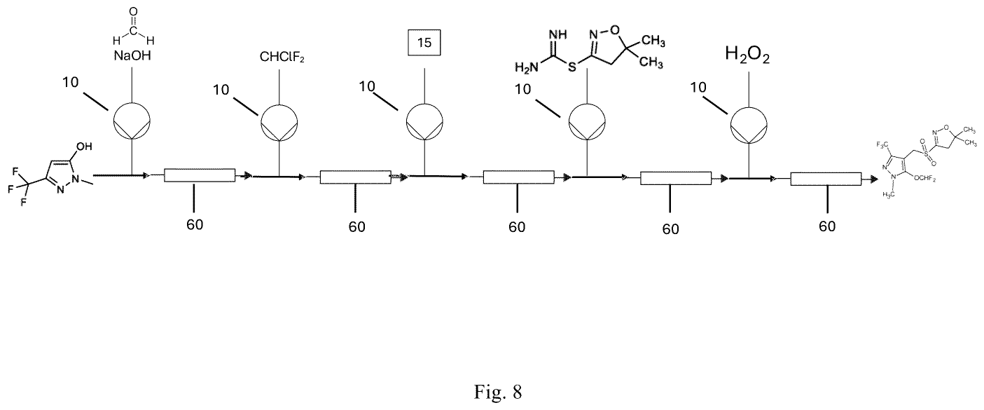

As another example of the present invention, the process proceeds as depicted in FIG. 8, and suitable solvent exchanges will be employed between each continuous-flow reactor where the solvent needs to be changed from one reaction to the next. Depending on the particular reaction conditions and the specifics of each reaction in the continuous-flow process, there may not be the need to employ a solvent exchange between each reaction. For instance, the reaction stream that contains compound of Formula II is passed through a solvent exchange system such that the compound of Formula II is dissolved in diglyme.

In certain embodiments wherein the continuous-flow process utilizes a multi-step process, acid or base can be continuously added to one or more of the continuous-flow reactors to control the pH within those reactors. For example, the intermediate 4-(((4,5-dihydro-5,5-dimethyl-3-isoxazolyl)thio)methyl)-1-methyl-3-(trifluoro-methyl)-1H-pyrazol-5-ol is more stable at low pH conditions. A continuous-flow process that uses 4-(((4,5-dihydro-5,5-dimethyl-3-isoxazolyl)thio)methyl)-1-methyl-3-(trifluoro-methyl)-1H-pyrazol-5-ol as one of the steps can improve reaction efficiency by continuously adding acid to maintain a low pH in the reaction medium.

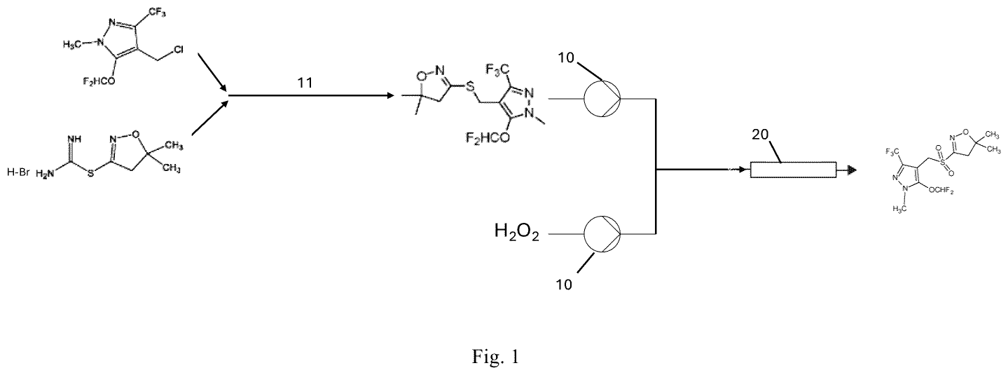

In another embodiment, the reaction proceeds according to FIG. 1 using (5,5-dimethyl(4,5- dihydroisoxazolo-3-yl)) thiocarboxamidine hydrobromide and 4-chloromethyl-5-difluoromethoxy-1-methyl-3-trifluoromethyl-pyrazole as starting materials. The starting materials are reacted with sodium hydroxide at 100° C. in a suitable solvent (such as methanol) in batch process 11 to form a compound of Formula II. A solution of the produced compound of Formula II is then prepared by dissolving the compound in a suitable solvent (such as preferably diglyme) and adding 0.1 equivalents of acetic acid, and the resulting solution is then pumped using a feed pump 10 (such as HPLC pumps), through a line which leads to a continuous-flow reactor 20 (such as a tubular reactor or coiled tubular reactor) held at 90-95° C. A solution of hydrogen peroxide (about 30% to about 50%) is pumped using feed pump 10 at a rate of 0.1 to 4 mL per minute through a second line which leads to the same continuous-flow reactor, in which the oxidation of compound of Formula II occurs. The reaction stream in the continuous-flow reactor has a retention time of approximately 2 to 4 hours. After the specified retention time, the reaction stream is collected for quenching with sodium bisulfite, with the pyroxasulfone product is obtained through standard filtration or crystallization.

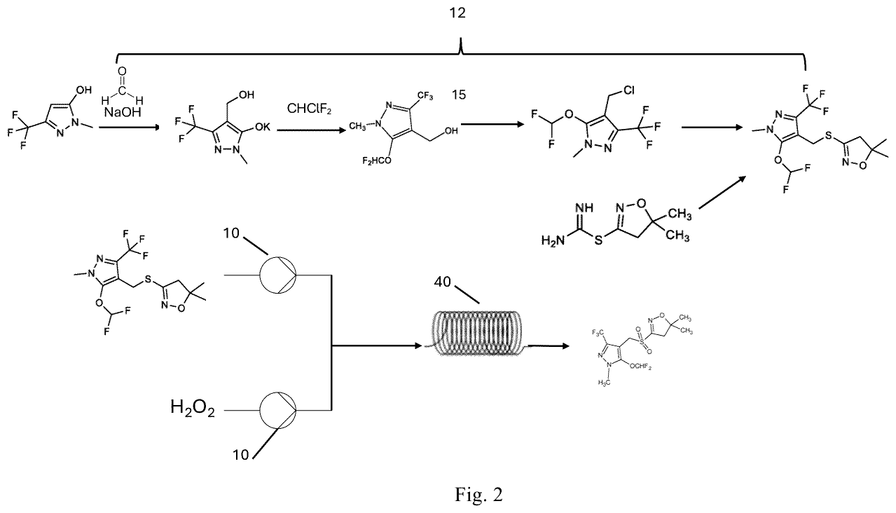

In another embodiment, the reaction proceeds according to FIG. 2 using (1-methyl-3-(trifluoromethyl)-1H-pyrazol-5-ol) as a starting material. (1-methyl-3-(trifluoromethyl)-1H-pyrazol-5-ol) is reacted with formaldehyde and sodium hydroxide to form 4-(hydroxymethyl)-1-methyl-3-(trifluoromethyl)-1H-pyrazol-5-olate. 4-(hydroxymethyl)-1-methyl-3-(trifluoromethyl)-1H-pyrazol-5-olate is then reacted with chlorodifluoromethane to form 5-(difluoromethoxy)-1-methyl-3-(trifluoromethyl)-1H-pyrazol-4-yl)methanol. 5-(difluoromethoxy)-1-methyl-3-(trifluoromethyl)-1H-pyrazol-4-yl)methanol is then reacted with a chlorinating agent to form 3-(((5-(difluoromethoxy)-1-methyl-3-trifluoromethyl)-1H-pyrazol-4-yl)methyl)thio)-5,5-dimethyl-4,5-dihydroisoxazole, which is then reacted with 5,5-dimethyl-4,5-dihydroisoxazol-3-yl-carbamimidothioate to form a compound of Formula II. All preceding steps 12 can be performed using either a batch process, or a continuous-flow process. The reaction medium containing a compound of Formula II dissolved in diglyme is pumped a using a feed pump 10 at a rate of 2 to 8 mL per minute through a first line which leads to a one liter coiled tubular reactor 50 held at 90-95° C. A solution of hydrogen peroxide (about 30% to about 50%) is pumped using a feed pump 10 at a rate of 0.1 to 4 mL per minute through a second line which leads to the same coiled tubular reactor 40, in which the oxidation of compound of Formula II occurs. The reaction medium in the coiled tubular reactor has a retention time of approximately 2 to 4 hours. After the specified retention time, the reaction medium is collected for quenching with sodium bisulfite, with the pyroxasulfone product is obtained through standard filtration or crystallization.

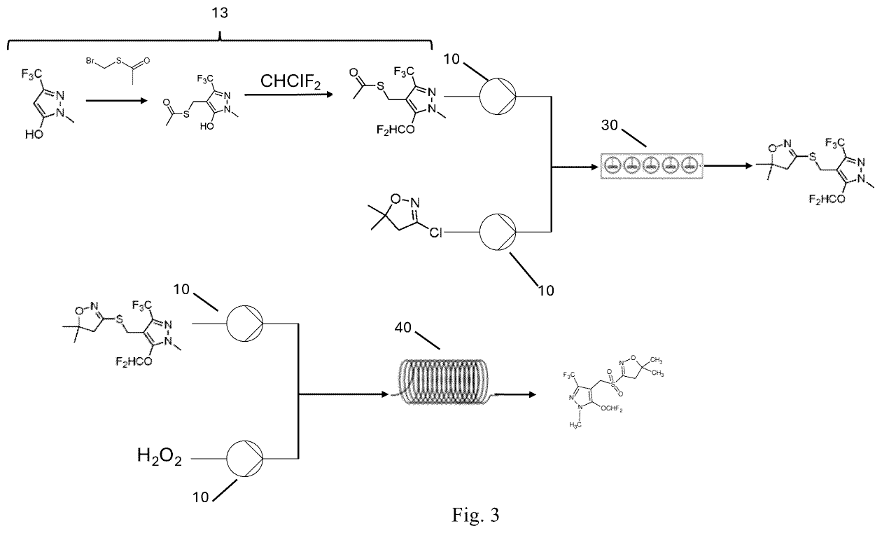

In another embodiment, the reaction proceeds according to FIG. 3 using 1-methyl-3-(trifluoromethyl)-1H-pyrazol-5-ol as a starting material that is reacted with S-(bromomethyl) ethanethioate to form thioacetic acid S-(5-hydroxy-l-methyl-3-trifluoromethyl-1H-pyrazol-4-ylmethyl) ester. The thioacetic acid S-(5-hydroxy-1-methyl-3-trifluoromethyl-1H-pyrazol-4-ylmethyl) ester is then reacted with chlorodifluoromethane at 80° C. to form thioacetic acid S-(5-difluoromethoxy-1-methyl-3-trifluoromethyl-1H-pyrazol-4-ylmethyl) ester. The preceding steps are performed as Batch Process 13. A first solution of thioacetic acid S-(5-difluoromethoxy-1-methyl-3-trifluoromethyl-1H-pyrazol-4-ylmethyl) ester is prepared (by dissolving in a suitable solvent, such as methanol). A second solution of 3-chloro-5,5-dimethyl-4,5-dihydroisoxazole is prepared (by dissolving in a suitable solvent, such as methanol). The two solutions are then pumped using feed pumps 10 (such as HPLC pumps) through separate lines into a 5-liter continuous-stirred tank reactor 30 that is held at 50° C. The reaction medium has a residence time within the continuous-stirred tank reactor 30 of four hours. The product of the CSTR step is a compound of Formula II. The reaction stream containing a compound of Formula II is then pumped using a feed pump 10 through a solvent exchange system such that the compound of Formula II is dissolved in diglyme and acetic acid. The reaction stream is then pumped using a feed pump 10 into a 1-liter coiled tubular reactor 40 that is preheated to a temperature of about 90 to 95° C. The pump rate for the solution of compound of Formula II is approximately 2 to 8 mL per minute. Simultaneously, a solution of hydrogen peroxide (about 30% to about 50%) is also pumped using a feed pump 10 into the coiled tubular reactor 40, at a rate of approximately 0.1 to 4 mL per minute. The reaction medium in the coiled tubular reactor has a retention time of approximately 2 to 4 hours. After the specified retention time, the reaction medium is collected for quenching with sodium bisulfite, with the pyroxasulfone product is obtained through standard filtration and crystallization.

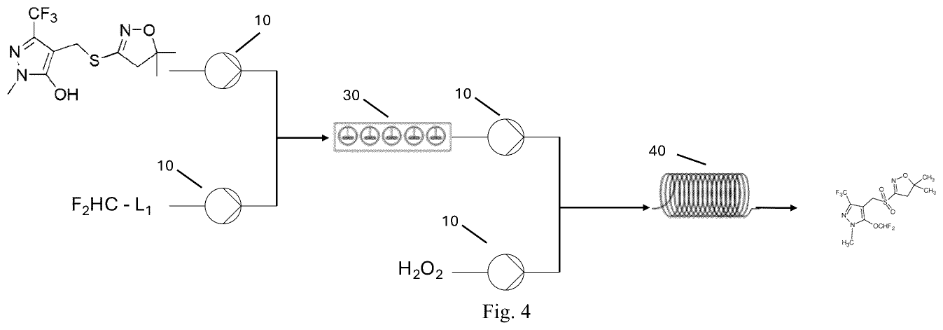

In another embodiment of the present invention, the process proceeds according to FIG. 4. In the first step, 4-(((4,5-dihydro-5,5-dimethyl-3-isoxazolyl)thio)methyl)-1-methyl-3-(trifluoro-methyl)-1H-pyrazol-5-ol and a fluorinated methyl halide (CHF₂-L₁) are used as the starting materials. A first solution of 4-(((4,5-dihydro-5,5-dimethyl-3-isoxazolyl)thio)methyl)-1-methyl-3-(trifluoro-methyl)-1H-pyrazol-5-ol is prepared (in a suitable solvent, such as water). A second solution of a fluorinated methyl halide (CHF₂-L₁) is prepared in a suitable solvent (such as acetonitrile). Then, both solutions are pumped using feed pumps 10 into a continuous-stirred tank reactor 30, is cooled to 10° C., with the reaction medium having a residence time of 4 hours. The product of the CSTR step is a compound of Formula II. The reaction stream containing compound of Formula II from the CSTR then flows to a solvent exchange system in which the solvent is exchanged for a mixture of diglyme and acetic acid. At the next stage, the compound of Formula II is pumped using a feed pump 10 into a one-liter coiled tubular reactor 40 that is preheated to a temperature of about 90 to 95° C. The pump rate for the compound of Formula II is approximately 2 to 8 mL per minute. Simultaneously, a solution of hydrogen peroxide (about 30% to about 50%) is also pumped using a feed pump 10 into the reactor, at a rate of approximately 0.1 to 4 mL per minute. The reaction medium in the reactor has a retention time of approximately 2 to 4 hours. After the specified retention time, the reaction medium is collected for quenching with sodium bisulfite, and then pyroxasulfone product is obtained through standard filtration and crystallization.

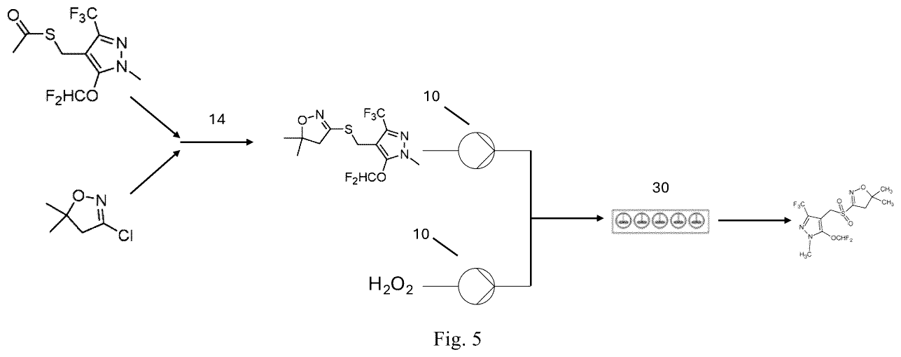

In another embodiment, the process proceeds according to FIG. 5 using 3-chloro-5,5-dimethyl-4,5-dihydroisoxazole and thioacetic acid S-(5-difluoromethoxy-1-methyl-3-trifluoromethyl-1H-pyrazol-4-ylmethyl)-ester as starting materials to prepare a compound of Formula II in a batch process 14. A solution of the compound of Formula II is prepared by dissolving the compound in a suitable solvent (such as diglyme) and adding 0.1 equivalents of acetic acid which is then pumped using a feed pump 10 (such as HPLC pumps), at rate of 10-20 mL per minute using a feed pump 10 through a line which leads to a 50-liter CSTR 30 held at 80° C. A solution of hydrogen peroxide (about 30% to about 50%) is pumped at a rate of 5 to 10 mL per minute through a second line which leads to the same reactor, in which the oxidation of compound of Formula II occurs. The reaction medium in the CSTR has a retention time of approximately 2 hours. After the specified retention time, the reaction medium is collected for quenching with sodium bisulfite, with the pyroxasulfone product is obtained through standard filtration and crystallization.

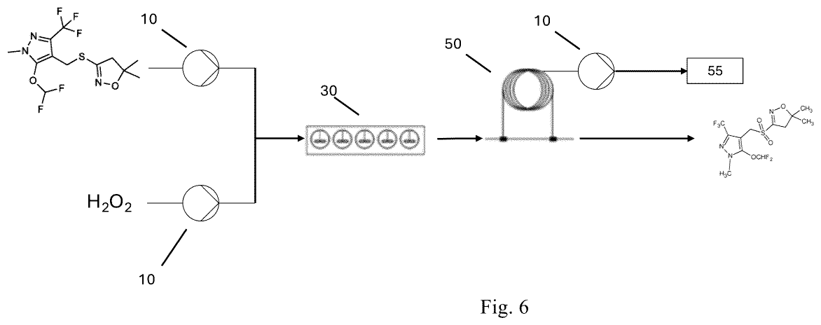

In another embodiment, the process proceeds according to FIG. 6 wherein a solution of compound of Formula II is prepared by dissolving compound of Formula II in a suitable solvent (such as diglyme) and adding 0.1 to 1.0 equivalents of acetic acid. The solution of compound of Formula II is pumped using a feed pump 10 (such as an HPLC pump) at a rate of 5-10 mL per minute through a first line to a 25-liter CSTR 30 heated to 90-95° C. The CSTR is heated using steel beads heated through induction heating. A solution of hydrogen peroxide (about 30% to about 50%) is pumped using a feed pump 10 at a rate of 2.5 to 5 mL per minute through a second line which leads to the same CSTR reactor 30, in which the oxidation of compound of Formula II occurs. The residence time of the reaction medium is approximately 2.5 hours. As the reaction medium exits the tubular reactor to be collected, the reaction medium passes through a sample loop 50 from which samples can be obtained using a feed pump 10 to be analyzed off-line using HPLC 55. After the specified residence time, the reaction medium is collected for quenching with sodium bisulfite, with the pyroxasulfone product is obtained through standard filtration and crystallization. After the specified retention time, the reaction medium is collected for quenching with sodium bisulfite, with the pyroxasulfone product is obtained through standard filtration and crystallization.

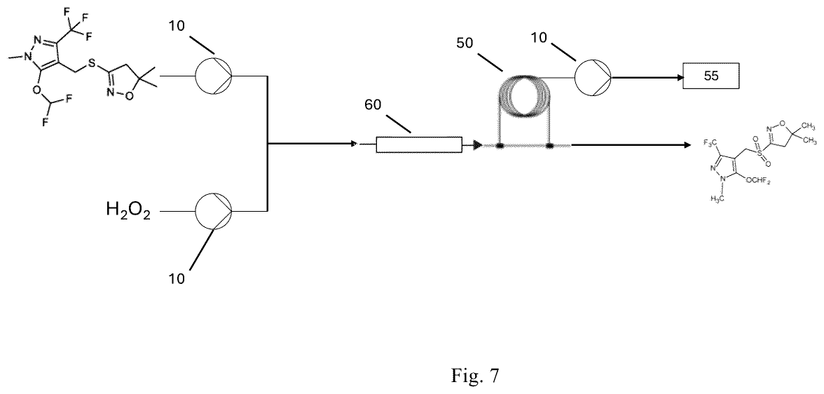

In another embodiment, the process proceeds according to FIG. 7. A solution of the compound of Formula II is prepared by dissolving the compound in a suitable solvent (such as diglyme) and adding 0.1 equivalents of acetic acid. The solution of compound of Formula II is then pumped at a rate of 4.0 g per minute using a feed pump 10 (such as HPLC pumps), through a line which leads to a tubular reactor 60 held at 90-95° C. A solution of hydrogen peroxide (about 30% to about 50%) is pumped using feed pump 10 at a rate of 0.8 g per minute through a second line which leads to the same tubular reactor 60, in which the oxidation of compound of Formula II occurs. The reaction medium in the tubular reactor has a residence time of 3 hours. As the reaction medium exits the tubular reactor to be collected, the reaction medium passes through a sample loop 50 from which samples can be pumped using a feed pump 10 to be analyzed off-line using HPLC 55. After the specified residence time, the reaction medium is collected for quenching with sodium bisulfite, with the pyroxasulfone product is obtained through standard filtration and crystallization.

In another embodiment, the process proceeds according to FIG. 8. A solution of (1-methyl-3-(trifluoromethyl)-1H-pyrazol-5-ol) is prepared by dissolving (1-methyl-3-(trifluoromethyl)-1H-pyrazol-5-ol) in a polar solvent (such as water). The solution is pumped through a line at a rate of 10 mL per minute to the first tubular reactor with a volume of 500 mL. A solution of formaldehyde and sodium hydroxide dissolved in water is simultaneously pumped into the first tubular reactor 60 using a feed pump 10 at a rate of 5 mL per minute. The first tubular reactor 60 is heated or cooled to a temperature of approximately 20 to 50° C. The reaction medium has a residence time in the first reactor of approximately 1 minute. The resulting reaction mixture is then pumped into the second tubular reaction at a rate of 15 mL per minute. The second tubular reactor 60 has a volume of 500 mL and is held at approximately 20 to 50° C. A solution of chlorodifluoromethane dissolved in acetonitrile is pumped using a feed pump 10 into the second tubular reactor at a rate of 5 mL per minute. The residence time of the reaction medium in the second reactor is approximately 5 to 10 minutes. The resulting reaction mixture is then pumped into the third tubular reactor 60 at a rate of approximately 20 mL per minute. The third tubular reactor has a volume of 500 mL and is held at approximately 20-50° C. A chlorinating agent 15 (such as thionyl chloride) is pumped into the third tubular reactor 60 using a feed pump 10 at a rate of 5 mL per minute. The residence time of the reaction medium in the third reactor is approximately 2 to 5 minutes. The resulting reaction mixture is then pumped into the fourth tubular reactor at a rate of 25 mL per minute. The fourth tubular reactor 60 has a volume of 500 mL and is held at approximately 100° C. A solution of 5,5-dimethyl-4,5-dihydroisoxazol-3-yl carbamimidothioate dissolved in water is pumped into the fourth tubular reactor 60 using a feed pump 10 at a rate of 5 mL per minute. The residence time of the reaction medium in the third tubular reactor 60 is approximately 1 to 5 minutes. The resulting reaction mixture is then pumped into the fifth tubular reactor at a rate of 30 mL per minute. The fifth tubular reactor has a volume of 50 L and is held at approximately 90-95° C. A mixture of diglyme and acetic acid is pumped through one line at a rate of 65 mL per minute into the fifth tubular reactor 60. Simultaneously, a solution of hydrogen peroxide (30-50%) is pumped through a second line using a feed pump 10 at a rate of 5 mL per minute into the fifth tubular reactor. The residence time of the reaction medium in the fifth tubular reactor is approximately 2 to 4 hours. After the specified residence time, the reaction medium is collected for quenching with sodium bisulfite, with the pyroxasulfone product is obtained through standard filtration and crystallization.

For each of the preceding embodiments, one or more steps of the complete process may be performed under continuous-flow conditions, and one or more steps may be performed under batch conditions. For instance, in one embodiment, all steps in the manufacturing process before the last step of oxidizing the compound of Formula II to form pyroxasulfone are performed as batch reactions, and then the compound of Formula II is converted to pyroxasulfone, under conditions described herein, in a continuous-flow process. In another embodiment, all steps in the manufacturing process can be performed under continuous-flow conditions by performing each step in a separate reactor such that the products flow from reactor to reactor.

A further embodiment of the continuous-flow process for making pyroxasufone comprises: (1) preparing a first solution of 1-methyl-3-(trifluoromethyl)-1H-pyrazol-5-ol and a base; (2) preparing a second solution comprising 5,5-dimethyl-4,5-dihydroisoxazol-3-yl-carbamimidothioate; (3) feeding via a first metering pump the solution of 1-methyl-3-(trifluoromethyl)-1H-pyrazol-5-ol with a base and feeding via a second metering pump a formaldehyde solution into a continuous-flow reactor (such as a microchannel reactor); (4) allowing the pumped reaction stream to proceed through the continuous-flow reactor for a specified time at a specified temperature; (5) continuously pumping the second solution comprising 5,5-dimethyl-4,5-dihydroisoxazol-3-yl-carbamimidothioate into a second continuous-flow reactor (such as a microchannel reactor) through a third metering pump and a base through a fourth metering pump into the second continuous-flow reactor according to a certain proportion, and reacting for a certain time at a certain temperature; (6) pumping (or allowing to flow) the outlet reaction streams from the first continuous-flow reactor and the second continuous-flow reactor into a third continuous-flow reactor (optionally with an intervening mixer or having a mixer within the third continuous-flow reactor) for a specified time at a specified temperature; (7) the reaction stream exiting the third continuous-flow reactor is pumped using a fifth metering pump into a fourth continuous-flow reactor; (8) Acid is continuously pumped into the fourth continuous flow reactor through a sixth metering pump according to a certain proportion to be mixed with materials at the outlet of the third continuous reactor, the mixture reacts for a certain time at a certain temperature. The solid-liquid mixture from the fourth continuous-flow reactor enters into a product receiving kettle, and the solid-liquid mixture is further fully mixed, centrifuged, washed and dried to obtain the 4-{[(5,5-dimethyl-4,5-dihydro-1,2-oxazol-3-yl)sulfanyl]methyl}-1-methyl-3-(trifluoromet-hyl)-1H-pyrazol-5-ol. 4-{[(5,5-dimethyl-4,5-dihydro-1,2-oxazol-3-yl)sulfanyl]methyl}-1-methyl-3-(trifluoromethyl)-1H-pyrazol-5-ol is then reacted with a solution of chlorodifluoromethane through a continuous or batch process to form the compound of Formula II. The compound of Formula II is then oxidized with hydrogen peroxide to form pyroxasulfone, as described herein (e.g., using a glyme as a solvent, preferably in the absence of a metal catalyst, preferably in the presence of a carboxylic acid (such as acetic acid, 0.1 equivalents), and optionally under continuous-flow conditions).

In the preceding embodiment, the molar ratio of 1-methyl-3-(trifluoromethyl)-1H-pyrazol-5-ol to the base (such as NaOH, KOH, Na₂CO₃ or other similar bases) may be from about 1 to about 4, preferably between 1.0 to 1.5, and the solution can be water, methanol, acetonitrile, or mixtures thereof.

In the preceding embodiment, the molar ratio of the 1-methyl-3-(trifluoromethyl)-1H-pyrazol-5-ol alkali solution to formaldehyde is about 1 to about 4, and the solution of formaldehyde that is used can be from about 0.1 mol/L to about 20 mol/L. The formaldehyde solution can be an aqueous solution or dissolved in an alkanol (such as methanol or ethanol).

In the preceding embodiment, the reaction temperature in the second continuous-flow reactor is from about −20° C. to about 50° C. The reaction time will vary and can be from about up to about thirty minutes.

In the preceding embodiment, in step (5), the molar ratio of the 5,5-dimethyl-4,5-dihydroisoxazol-3-yl-carbamimidothioate is about 1 to about 4, with a molar concentration of the 5,5-dimethyl-4,5-dihydroisoxazol-3-yl-carbamimidothioate solution is 0.1-5 mol/L. The base used can be NaOH or others recited above, with the solution comprising water, methanol, acetonitrile, and mixtures thereof.

In the preceding embodiment, the reaction temperature in the third continuous-flow reactor is about −20° C. to 50° C., with a reaction time of about 1 minute to about 30 minutes.

In the preceding embodiment, the reaction temperature in fourth continuous reactor is −20° C. to 50° C., preferably −10° C. to 20° C., and the reaction time is 0.1 to 30 min, preferably 1 to 10 min.

In the preceding embodiment the molar ratio of 1-methyl-3-(trifluoromethyl)-1H-pyrazol-5-ol to 5,5-dimethyl-4,5-dihydroisoxazol-3-yl-carbamimidothioate and the acid in step (8) is from about 1:1 to about 1:5. The acid can be one or more of hydrochloric acid, sulfuric acid and phosphoric acid, preferably hydrochloric acid, and the molar concentration of the acid is from about 0.1 to about 5 mol/L.

In the preceding embodiment, the molar ratio of 1-methyl-3-(trifluoromethyl)-1H-pyrazol-5-ol to formaldehyde to 5,5-dimethyl-4,5-dihydroisoxazol-3-yl-carbamimidothioate is from about 1:3 to about 3:1.

In yet a further aspect of the present invention, the continuous-flow process as described herein is performed in a semi-automated or fully automated manner. When the continuous-flow process is performed in a semi-automated or fully automated manner, one or more aspects or parameters of the reaction process are adjusted automatically during the reaction in order to optimize reaction conditions. The automated adjustments to the reaction parameters are typically based on sensing reaction conditions, such as temperature or percent completion of the reaction immediately after the reaction mixture leaves the reactor. For instance, in-line reaction monitoring can be used. “In-line” refers to a platform configuration whereby the analytics are connected to the reactor, and the reaction mixture is analyzed right after leaving the reactor. In-line high-resolution liquid chromatography-MS for real-time analysis of the automated continuous-flow system can be used in certain embodiments. In-line React-IR monitoring using FTIR spectroscopy, e.g., to measure the amount of pyroxasulfone or impurities can be used in other embodiments. The data from the real-time analysis can be used by the control system to automatically adjust the reaction conditions of the continuous-flow reactor based on preset parameters. For instance, if the percent conversion to pyroxasulfone is below a certain percentage, such as 95%, one or more reaction conditions can automatically be adjusted, such as increasing the temperature of the reactor. After the specified residence time, the reaction medium is collected for quenching with sodium bisulfite, with the pyroxasulfone product is obtained through standard filtration and crystallization.

Alternatively, if the sensor indicates that a byproduct or impurity is above a certain level, then the reaction conditions can be automatically adjusted to ensure that the byproduct or impurity does not exceed a specified level.

In an embodiment of the present invention, the monitoring the reaction process involves collecting the reaction stream exiting the reactor (for example, by taking aliquots or using a sample loop) for inline or atline analysis using HPLC. HPLC analysis is then used to measure the amounts of sulfoxide impurity of Formula III and/or sulfonic acid impurity of Formula IV. Depending on the amount of impurities present, the control system can automatically adjust the reaction conditions of the continuous-flow reactor based on certain preset parameters such as temperature or residence time to minimize the amount of impurities produced.

In another aspect of the present invention, the various methods of preparing pyroxasulfone, as described herein, are coupled with a continuous crystallization step in order to obtain the final pyroxasulfone product. Any one of various apparatuses can be used for the continuous crystallization of pyroxasulfone. Such an apparatus can be a mixed suspension mixed product removal (“MSMPR”) crystallizer; a PFC or a tubular crystallizer; a multistage COBC system; a tubular crystallizer with segmented flow; and a CFI crystallizer in which, due to the centrifugal direction, a homogeneous suspension flow can be achieved at a suitable flow velocity.

A MSMPR crystallizer is similar in structure to continuous-stirred tank reactor. The MSMPR is typically a jacketed vessel with agitation that is designed to achieve homogeneous mixing at any given time. Materials are fed into the tank through one inlet or multiple inlets continuously and removed from the outlet.

In other embodiments, the present invention further comprises continuous crystallization that may comprise continuous spatially distributed diafiltration, parallel agitated bed crystallization, continuous spatially distributed distillation, and concentric annular liquid-liquid extraction.

### Herbicidal Compositions

Another aspect of the present invention is an agrochemical composition or formulation comprising pyroxasulfone made according to the novel process described herein and one or more suitable excipients. Agriculturally and herbicidally suitable excipients are preferred. Also disclosed are methods of making such an agrochemical composition or formulation comprising pyroxasulfone made according to any of the embodiments of the novel process described herein and one or more suitable excipients.

Suitable excipients are preferably one or more agrochemically acceptable excipients. In an embodiment, agriculturally acceptable excipient or carriers can be selected from one or more diluents, emulsifiers, fillers, anti-foaming agents, thickening agents, anti-freezing agents, freezing agents, surfactants, preservatives, coloring agents, pH-adjusting agents, dispersing agents, wetting agents, and solvents. It should be appreciated, however, that other agriculturally acceptable excipients, as known to a person skilled in the art, may be used to serve its intended purpose. In an embodiment, the agriculturally acceptable excipients are present in an amount ranging from 0.01% to 90% by weight of the total composition, with a preferred range of from about 10% to about 30%.

According to another embodiment, the present herbicide composition further comprising at least one additional herbicide.

In an embodiment, the additional herbicide is a triazinone herbicide. In an embodiment, the herbicidal composition comprising a combination of pyroxasulfone prepared according to the present process and a triazinone herbicide. In an embodiment, the triazinone herbicide is selected from the group of ametridione, amibuzin, ethiozin, hexazinone, isomethiozin, metamitron, metribuzin, or trifludimoxazin. In an embodiment, the triazinone herbicide is metribuzin. According to an embodiment, the present invention provides herbicidal composition comprising combination of pyroxasulfone prepared according to present process and metribuzin.

According to another embodiment of the invention, the present compositions are formulated as water-dispersible granules. When formulated as water-dispersible granules, the pyroxasulfone compositions obtained according to present invention are capable of dispersing quickly in water. According to an embodiment, the compositions of pyroxasulfone obtained according to present invention leads to optimum suspensibility while dispersed in water.

According to an embodiment, the composition prepared is a water dispersible granule comprising pyroxasulfone, at least one dispersing agent and at least one wetting agent. According to an embodiment, the dispersing agent/wetting agent used is selected from, but not limited to, group comprising of anionic, cationic or zwitterionic and/or non-ionic surface-active compounds (surfactants) or combinations thereof, preferably anionic surfactant is used.

Examples of anionic surfactants include: anionic derivatives of fatty alcohols having 10-24 carbon atoms in the form of ether carboxylates, sulfonates, sulfates, and phosphates, and their inorganic salts (e.g., alkali metal and alkaline earth metal salts) and organic salts (e.g., salts based on amine or alkanolamine); anionic derivatives of copolymers consisting of EO (ethylene oxide), PO (propylene oxide) and/or BO (butylene oxide) units, in the form of ether carboxylates, sulfonates, sulfates, and phosphates, and their inorganic salts (e.g., alkali metal and alkaline earth metal salts) and organic salts (e.g., salts based on amine or alkanolamine) or acrylic/styrene copolymers, methacrylic copolymers; linear (C₈-C₁₅) alcohol derivative and their salts; alkyl aryl sulfonates including but not limited to alkyl benzenesulfonates; alkyl naphthalene sulfonates and salts thereof and salts of ligninsulfonic acid; derivatives of alkylene oxide adducts of alcohols, in the form of ether carboxylates, sulfonates, sulfates and phosphates, and their inorganic salts (e.g., alkali metal and alkaline earth metal salts) and organic salts (e.g., salts based on amine or alkanolamine); anionic derivatives of fatty acid alkoxylates, in the form of ether carboxylates, sulfonates, sulfates and phosphates, and their inorganic salts (e.g., alkali metal and alkaline earth metal salts) and organic salts (e.g., salts based on amine or alkanolamine); alkyl ether phosphate, alkyl sulfosuccinate mono ester and diester salts.

Preferably, sulfosuccinates and their derivatives/salts; acrylic/styrene copolymers; salts of lignin sulfonic acid are used.

According to an embodiment, the composition may further comprise a defoamer. The defoamer used is selected from, but not limited to, group comprising of aqueous emulsion with polysiloxane and emulsifier, silicone oil and magnesium stearate or a suitable combination thereof.

According to an embodiment, the water-dispersible granule comprising pyroxasulfone is prepared by a process comprising: a) mixing pyroxasulfone made according the present invention with one or more wetting agents and one or more dispersing agents as required; b) milling the mixture in a suitable equipment to obtain a powder having a particle size D90<15 pm; and c) granulating the powder by suitable means and drying the granules obtained.

In order to better illustrate the objectives, technical details, and advantages of the present disclosure, the disclosure will be described below in detail in conjunction with embodiments.

### Example 1

### Preparation of Pyroxasulfone Using Diglyme as a Solvent

To one mole of a compound of Formula II, in a 3-necked round bottomed flask, are 300 to 400 grams of diglyme at 25 to 30° C. added under constant agitation. Three to five moles of aqueous H₂O₂ (30% to 50% strength) per mole of the compound of Formula II are introduced into the reaction mixture via an addition funnel over a period of two to three hours at 30 to 50° C. under constant agitation. After completion of the addition of H₂O₂, the reaction temperature is gradually increased to 90 to 95° C. under constant agitation. The reaction mixture is held at this temperature for a period of 4 to 6 hours with HPLC analyses of aliquot samples in order to monitor reaction kinetics (consumption of the compound of Formula II, and the formation of the desired pyroxasulfone and possible monitoring of the sulfoxide intermediate). The reaction is deemed to be complete when the 6-hour sample indicates absence of the compound of Formula II and less than 0.1% of the sulfoxide intermediate (compound of Formula III). The reaction mixture is then cooled to 25 to 30° C. under agitation prior to filtration. Pyroxasulfone is isolated via filtration and is washed with 100 grams of water prior to air drying. The product is of greater than 99% purity via HPLC analysis with undetectable amounts of the compounds of Formula II and III. The mother liquor (solvent diglyme containing water and excess H₂O₂) is retained for treatment prior to recovery/recycle to subsequent batch.

### Example 2

### Preparation of Pyroxasulfone Using Diglyme as a Solvent

The process as per Example 1 is repeated wherein the reaction temperature is reduced to the 65 to 70° C. range under identical stoichiometric conditions. The reaction is deemed complete in 7 hours as per HPLC analysis. The product, pyroxasulfone, is isolated via filtration and washed with water, as per Example 1. HPLC analysis will indicate a product of purity of 99.2% or greater, with undetectable levels of the compounds of Formula II or III. The mother liquor is retained for treatment prior to recovery/recycle to subsequent batch.

### Example 3

Treatment of Mother Liquor Containing Solvent Diglyme, Water, and Excess H₂O₂

Sodium bisulfite (10 grams per 100 grams of mother liquor) is added to the mother liquor at 25° C. to 30° C. with agitation. Excess H2O2 present in mother liquor is thereby reduced within two hours. Lower boiling water is distilled off from the mother liquor, to leave behind the diglyme for recycle to a subsequent batch after a filtration step to remove any suspended inorganic sodium salts.

### Example 4

### Larger-Scale Preparation of Pyroxasulfone Using Diglyme as a Solvent

The reaction according to Example 2 is performed using identical stoichiometric conditions, except that the reaction is performed starting with 100 moles of the compound of Formula II.

### Example 5

### Continuous Flow Preparation of Pyroxasulfone Using a Tubular Reactor and Diglyme as a Solvent

A solution of the compound of Formula II is prepared in a first container/vessel comprising 0.3 mol compound of Formula II, 462 g of diglyme, and 0.03 mol of acetic acid. The solution for the compound of Formula II is mixed thoroughly. A second solution of 35% hydrogen peroxide is prepared in a second container/vessel. The tubular reactor is then heated to 95° C. A feed pump is then used to pump the solution comprising compound of Formula II at a rate of 4.0 g/min, and another feed pump is used to pump 35% hydrogen peroxide at a rate of 0.8 g/min. The reactants are then kept in the reactor to undergo continuous oxidation, holding the temperature between 90° C. and 95° C. The reactants are then circulated through the reactor for a retention time of 180 minutes. The resulting reaction mixture containing crude pyroxasulfone is then collected as it exits the tubular reactor, and excess hydrogen peroxide is quenched with sodium sulfite. The crude pyroxasulfone is then washed with water and cooled to 0° C., before purification through filtration and drying to obtain pyroxasulfone at 99% purity.

### Example 6

### Continuous Flow Preparation of Pyroxasulfone Using a Tubular Reactor and Dimethoxyethane as a Solvent

A solution of the compound of Formula II is prepared in a first container/vessel comprising 0.3 mol compound of Formula II, 462 g of dimethoxyethane, and 0.03 mol of acetic acid. The solution for the compound of Formula II is mixed thoroughly. A second solution of 35% hydrogen peroxide is prepared in a second container/vessel. The tubular reactor is then heated to 95° C. A feed pump is then used to pump the solution comprising compound of Formula II at a rate of 4.0 g/min, and another feed pump is used to pump 35% hydrogen peroxide at a rate of 0.8 g/min. The reactants are then kept in the reactor to undergo continuous oxidation, holding the temperature between 90° C. and 95° C. The reactants are then circulated through the reactor for a retention time of 180 minutes. The resulting reaction mixture containing crude pyroxasulfone is then collected as it exits the tubular reactor, and excess hydrogen peroxide is quenched with sodium sulfite. The crude pyroxasulfone is then washed with water and cooled to 0° C., before purification through filtration and drying to obtain pyroxasulfone at 99% purity.

### Example 7

### Continuous Flow Preparation of Pyroxasulfone Using a Tubular Reactor and Continuous Crystallization

A solution of the compound of Formula II is prepared in a first container/vessel comprising 0.3 mol compound of Formula II, 462 g of diglyme, and 0.03 mol of acetic acid. The solution for the compound of Formula II is mixed thoroughly. A second solution of 35% hydrogen peroxide is prepared in a second container/vessel. The tubular reactor is then heated to 95° C. A feed pump is then used to pump the solution comprising compound of Formula II at a rate of 4.0 g/min, and another feed pump is used to pump 35% hydrogen peroxide at a rate of 0.8 g/min. The reactants are then kept in the reactor to undergo continuous oxidation, holding the temperature between 90° C. and 95° C. The reactants are then circulated through the reactor for a retention time of 180 minutes. The resulting reaction mixture containing crude pyroxasulfone is then collected as it exits the tubular reactor, and excess hydrogen peroxide is quenched with sodium sulfite.

After quenching, the reaction stream is treated with a tubular crystallizer (with an inner diameter of less than 10 cm and a length of greater than 1 meter).

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

Having described what is considered the best form presently contemplated for embodying the present invention, various alterations, modifications, and/or alternative applications of the invention will be promptly apparent to those skilled in the art. Therefore, it is to be understood that the present invention is not limited to the practical aspects of the actual preferred embodiments hereby described and that any such modifications and variations must be considered as being within the spirit and the scope of the invention, as described in the above description.

What is claimed is:

1. A continuous-flow process for the preparation of pyroxasulfone (Formula I) or a salt thereof comprising:
a. preparing a first solution comprising a compound of Formula II and a carboxylic acid in a catalytic amount, wherein the first solution comprises one or more glymes as a solvent;
b. preparing a second solution comprising an oxidizing agent;
c. charging said first solution through a first feed line into a continuous-flow reactor unit, wherein the first feed line is fluidically connected with the continuous-flow reactor unit;
d. charging said second solution through a second feed line into the continuous-flow reactor, wherein the second feed line is fluidically connected with the continuous-flow reactor unit;
e. providing one or more reaction conditions in the continuous-flow reactor such that the first solution and the second solution interact, thereby effecting the oxidation of the compound of Formula II into pyroxasulfone, wherein said effecting the oxidation occurs in the absence of a metal catalyst; and
f. receiving the solution containing pyroxasulfone and then isolating the pyroxasulfone in solid form.

2. The process according to claim 1, wherein the glyme is diglyme.

3. The process according to claim 1, wherein the carboxylic acid is acetic acid.

4. The process according to claim 3, wherein the oxidizing agent is hydrogen peroxide at a concentration of 30% to 50%.

5. The process according to claim where, wherein the reaction is performed in the absence of a metal catalyst.

6. The process according to claim 1, wherein the continuous-flow reactor unit is heated so that the reaction stream within the reactor unit is heated to about 80° C. to about 100° C.

7. The process according to claim 6, wherein the reaction is held at about 90° C. to 95° C. for about four to about six hours.

8. The process according to claim 1, wherein the reactor is a tubular reactor.

9. The process according to claim 9, wherein the residence time of the reactants in the process is between two hours to four hours.

10. The process according to claim 9, wherein the pressure of the reaction vessel is from about 1 bar to about 10 bar.

11. The process according to claim 1, wherein the purity of pyroxasulfone is greater than 99% as measured by HPLC.

12. A process for producing pyroxasulfone by continuous-flow process comprising:
a) charging a first solution comprising a compound of Formula II, a carboxylic acid, and one or more suitable solvents through a first feed line into a continuous-flow reactor unit, in a continuous flow;
b) charging a second solution comprising an oxidizing agent and one or more suitable solvents through a second feed line into a reactor unit, in a continuous flow; and
c) oxidizing the compound of Formula II with the oxidizing agent in said reactor to form a reaction stream comprising pyroxasulfone, wherein said oxidizing is performed at a temperature of about 85° C. to about 100° C. and wherein the residence time in the continuous-flow reactor unit is from about 2 hours to about 5 hours.

13. The process according to claim 12, further comprising the addition of a metal catalyst selected from a tungsten catalyst, a molybdenum catalyst, a titanium catalyst, a zirconium catalyst, and mixtures thereof, and wherein said metal catalyst is added as a separate solution to the reactor unit.

14. The process according to claim 13, wherein said metal catalyst is a sodium tungstate.

15. An herbicidal composition comprising pyroxasulfone or salt thereof made according to claim 12 and one or more herbicidally acceptable excipients.
